(19) United States
(12) Patent Application Publication
(10) Pub. No.: US 2006/0019265 A1
(43) Pub. Date: Jan. 26, 2006

(54) Transmission-based luminescent detection systems

(75) Inventors: Xuedong Song, Roswell, GA (US); Michael Knotts, Roswell, GA (US); David Samuel Cohen, Alpharetta, GA (US); Shawn Ray Feaster, Duluth, GA (US); Zdravko Savov Atanassov, Alpharetta, GA (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 11/020,647

(22) Filed: Dec. 22, 2004

Related U.S. Application Data
(60) Provisional application No. 60/608,941, filed on Apr. 30, 2004.

(51) Int. Cl. C12Q 1/68; C12M 1/34; G01N 33/558

(52) U.S. Cl. 435/6; 435/287.2; 436/514

(57) ABSTRACT

A luminescent detection system that employs transmission-based detection is provided for use with a chromatographic-based assay device. Unlike conventional systems, the detection system of the present invention is portable, simple to use, and inexpensive. For example, the system may be selectively controlled to reduce reliance on expensive optical components, such as monochromators or narrow emission bandwidth optical filters. In addition, the detection system is also capable of eliminating background interference from many sources, such as scattered light and autofluorescence, which have often plagued conventional fluorescent detection systems.

### RELATED APPLICATIONS

The present application claims priority to a provisional application having Ser. No. 60/608,941, which was filed on Mar. 30, 2004.

### BACKGROUND OF THE INVENTION

Fluorescent detection techniques have been employed to determine the presence or absence of an analyte. For example, conventional fluorescence readers utilize an illumination source that causes fluorescent labels to emit photons at a certain wavelength. A detector registers the emission photons and produces a recordable output, usually as an electrical signal or a photographic image. In addition, the readers often utilize one or more optical elements to help focus, shape, or attenuate the transmitted fluorescent signals in a desired manner. For example, optical filters are sometimes utilized to isolate the emission photons from the excitation photons.

However, one problem with conventional fluorescent detection systems is that they utilize very complex optical elements, and thus are often bulky, non-portable, and expensive. In addition, some conventional optical detection systems are also problematic when used in conjunction with assay devices that contain a chromatographic medium, such as a porous membrane. For example, in a membrane-based device, the concentration of the analyte is reduced because it is diluted by a liquid that can flow through the porous membrane. Unfortunately, background interference becomes increasingly problematic at such low analyte concentrations because the intensity to be detected is relatively low. Because the structure of the membrane also tends to reflect the emitted light, the ability of the detector to accurately measure the intensity of the labeled analyte is substantially reduced. In fact, the intensity of the emitted signal is typically three to four orders of magnitude smaller than the excitation light reflected by the porous membrane.

As such, a need currently exists for an improved technique for determining the presence or absence of an analyte within a test sample. In particular, a need exists for a simple, inexpensive, and effective luminescent detection system that utilizes a chromatographic-based assay device.

### SUMMARY OF THE INVENTION

In accordance with one embodiment of the present invention, a luminescent (e.g., fluorescent, phosphorescent, etc.) detection system is disclosed for detecting the presence or quantity of an analyte residing in a test sample. The system comprises an assay device that includes a chromatographic medium in communication with luminescent detection probes. The luminescent detection probes are capable of emitting a detection signal. The system further comprises an illumination source and a detector. The illumination source is capable of providing electromagnetic radiation that excites the luminescent detection probes to emit the detection signal. The detector is capable of registering the detection signal emitted by the luminescent detection probes. The illumination source and detector are positioned on opposing sides of the assay device so that the chromatographic medium is positioned in the electromagnetic radiation path defined between the illumination source and detector. The chromatographic medium is transmissive to the electromagnetic radiation and the detection signal.

In accordance with another embodiment of the present invention, a luminescent detection system is disclosed for detecting the presence or quantity of an analyte residing in a test sample. The system comprises an assay device that includes a porous membrane carried by a support. The porous membrane is in communication with luminescent detection probes, which are capable of emitting a detection signal. The system further comprises an illumination source and a time-gated detector. The illumination source is capable of providing pulsed electromagnetic radiation that excites the luminescent detection probes to emit the detection signal. Likewise, the time-gated detector is capable of registering the detection signal emitted by the luminescent detection probes. The illumination source and detector are positioned on opposing sides of the assay device so that the porous membrane is positioned in the electromagnetic radiation path defined between the illumination source and detector. The porous membrane and support are transmissive to the electromagnetic radiation and detection signal.

In accordance with still another embodiment of the present invention, a method is disclosed for detecting the presence or quantity of an analyte residing in a test sample with an assay device. The assay device comprises a porous membrane that defines a detection zone, the porous membrane being in communication with luminescent detection probes. The method comprises contacting the test sample with the assay device, wherein at least a portion of the luminescent detection probes become immobilized within the detection zone. Electromagnetic radiation is pulsed onto the detection zone, thereby exciting the luminescent detection probes to emit a detection signal that is transmitted through the porous membrane. The intensity of the transmitted signal is measured. In one embodiment, for example, the amount of the analyte within the test sample is proportional to the intensity of the transmitted detection signal. If desired, a period of time may elapse between a pulse of electromagnetic radiation and measurement of the detection signal.

Other features and aspects of the present invention are discussed in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figure in which:

FIG. 1 is a schematic illustration of one embodiment of a luminescent detection system of the present invention;

FIG. 2 is a cross-sectional view of an electroluminescent (EL) device that may be used in one embodiment of the present invention;

FIG. 3 schematically illustrates various embodiments of the luminescent detection system of the present invention, in which FIG. 3a illustrates an embodiment in which the illumination source and detector are spaced relatively distant from the assay device; FIG. 3b illustrates the embodiment of FIG. 3a in which an illumination lens and a detection lens are also used to focus light to and from the assay device; FIG. 3c illustrates the embodiment of FIG. 3b in which the illumination lens is removed and the illumination source is moved closer to the assay device; and FIG. 3d illustrates the embodiment of FIG. 3c in which the detection lens is removed and the detector is moved closer to the assay device;

FIG. 4 is a schematic diagram of one embodiment of a luminescence reader that may be used in the present invention, including representative electronic components thereof;

FIG. 5 is a schematic diagram of another embodiment of a luminescence reader that may be used in the present invention, including representative electronic components thereof;

FIG. 6 is a schematic diagram of still another embodiment of a luminescence reader that may be used in the present invention, including representative electronic components thereof;

FIG. 7 is a schematic illustration of another embodiment of a luminescent detection system of the present invention, which employs an EL illumination source;

FIG. 8 is a perspective view of still another embodiment of a luminescent detection system of the present invention, which employs an LED-based illumination source and a photodiode-based detector;

FIG. 9 is a cross-sectional view of the luminescent detection system shown in FIG. 8;

FIG. 10 graphically depicts the results of Example 1, in which the phosphorescent signal is plotted versus time (microseconds);

FIG. 11 graphically depicts the results of Example 1, in which the dose response is plotted versus the amount of phosphorescent particles (nanograms);

FIG. 12 graphically depicts the results of Example 2, in which the phosphorescent signal is plotted versus time (microseconds);

FIG. 13 graphically depicts the results of Example 2, in which the dose response is plotted versus the amount of phosphorescent particles (nanograms);

FIG. 14 graphically depicts the results of Example 3, in which the dose response is plotted versus the amount of phosphorescent particles (nanograms);

and

FIG. 15 graphically depicts the fluorescence spectrum obtained in Example 4, in which intensity is plotted versus wavelength.

Repeat use of reference characters in the present specification and drawings is intended to represent same or analogous features or elements of the invention.

### DETAILED DESCRIPTION OF REPRESENTATIVE EMBODIMENTS

### Definitions

As used herein, the term “analyte” generally refers to a substance to be detected. For instance, analytes may include antigenic substances, haptens, antibodies, and combinations thereof. Analytes include, but are not limited to, toxins, organic compounds, proteins, peptides, microorganisms, amino acids, nucleic acids, hormones, steroids, vitamins, drugs (including those administered for therapeutic purposes as well as those administered for illicit purposes), drug intermediaries or byproducts, bacteria, virus particles and metabolites of or antibodies to any of the above substances. Specific examples of some analytes include ferritin; creatinine kinase MB (CK-MB); digoxin; phenytoin; phenobarbitol; carbamazepine; vancomycin; gentamycin; theophylline; valproic acid; quinidine; luteinizing hormone (LH); follicle stimulating hormone (FSH); estradiol, progesterone; C-reactive protein; lipocalins; IgE antibodies; cytokines; vitamin B2 micro-globulin; glycated hemoglobin (Gly. Hb); cortisol; digitoxin; N-acetylprocainamide (NAPA); procainamide; antibodies to rubella, such as rubella-IgG and rubella IgM; antibodies to toxoplasmosis, such as toxoplasmosis IgG (Toxo-IgG) and toxoplasmosis IgM (Toxo-IgM); testosterone; salicylates; acetaminophen; hepatitis B virus surface antigen (HBsAg); antibodies to hepatitis B core antigen, such as anti-hepatitis B core antigen IgG and IgM (Anti-HBC); human immune deficiency virus 1 and 2 (HIV 1 and 2); human T-cell leukemia virus 1 and 2 (HTLV); hepatitis B e antigen (HBeAg); antibodies to hepatitis B e antigen (Anti-HBe); influenza virus; thyroid stimulating hormone (TSH); thyroxine (T4); total triiodothyronine (Total T3); free triiodothyronine (Free T3); carcinoembryoic antigen (CEA); lipoproteins, cholesterol, and triglycerides; and alpha fetoprotein (AFP). Drugs of abuse and controlled substances include, but are not intended to be limited to, amphetamine; methamphetamine; barbiturates, such as amobarbital, secobarbital, pentobarbital, phenobarbital, and barbital; benzodiazepines, such as librium and valium; cannabinoids, such as hashish and marijuana; cocaine; fentanyl; LSD; methaqualone; opiates, such as heroin, morphine, codeine, hydromorphone, hydrocodone, methadone, oxycodone, oxymorphone and opium; phencyclidine; and propoxyhene. Other potential analytes may be described in U.S. Pat. No. 6,436,651 to Everhart. et al. and U.S. Pat. No. 4,366,241 to Tom et al.

As used herein, the term “test sample” generally refers to a biological material suspected of containing the analyte. The test sample may be derived from any biological source, such as a physiological fluid, including, blood, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid, semen, and so forth. Besides physiological fluids, other liquid samples may be used such as water, food products, and so forth, for the performance of environmental or food production assays. In addition, a solid material suspected of containing the analyte may be used as the test sample. The test sample may be used directly as obtained from the biological source or following a pretreatment to modify the character of the sample. For example, such pretreatment may include preparing plasma from blood, diluting viscous fluids, and so forth. Methods of pretreatment may also involve filtration, precipitation, dilution, distillation, mixing, concentration, inactivation of interfering components, the addition of reagents, lysing, etc. Moreover, it may also be beneficial to modify a solid test sample to form a liquid medium or to release the analyte.

### DETAILED DESCRIPTION

Reference now will be made in detail to various embodiments of the invention, one or more examples of which are set forth below. Each example is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention covers such modifications and variations as come within the scope of the appended claims and their equivalents.

In general, the present invention is directed to a system that employs transmission-mode luminescence detection techniques in conjunction with a chromatographic-based assay device. Unlike conventional systems, the detection system of the present invention is portable, simple to use, and inexpensive. For example, the system may be selectively controlled to reduce reliance on expensive optical components, such as monochromators or narrow emission bandwidth optical filters. In addition, the detection system is also capable of eliminating background interference from many sources, such as scattered light and autofluorescence, which have often plagued conventional fluorescent detection systems.

I. Assay Device

Generally speaking, the assay device employed in the present invention is configured to perform a heterogeneous immunoassay. A heterogeneous assay is an assay in which uncomplexed labeled species are separated from complexed labeled species. Separation may be carried out by physical separation, e.g., by transferring one of the species to another reaction vessel, filtration, centrifugation, chromatography, solid phase capture, magnetic separation, and so forth, and may include one or more washing steps. The separation may also be nonphysical in that no transfer of one or both of the species is conducted, but the species are separated from one another in situ. In one particular embodiment, for example, a heterogeneous immunoassay is utilized. Such immunoassays utilize mechanisms of the immune systems, wherein antibodies are produced in response to the presence of antigens that are pathogenic or foreign to the organisms. These antibodies and antigens, i.e., immunoreactants, are capable of binding with one another, thereby causing a highly specific reaction mechanism that may be used to determine the presence or concentration of that particular antigen in a fluid test sample.

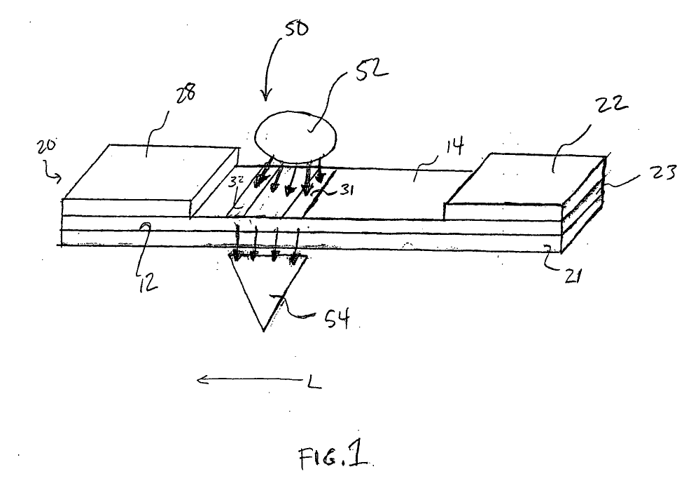

Referring to FIG. 1, for example, one embodiment of a chromatographic-based assay device 20 that is configured to perform a heterogeneous immunoassay will now be described in more detail. As shown, the assay device 20 contains a chromatographic medium 23 having a first surface 12 and an opposing second surface 14. The first surface 12 of the medium 23 is positioned adjacent to a support 21. The chromatographic medium 23 is generally made from a material through which the test sample is capable of passing, such as a fluidic channel, porous membrane, etc. Likewise, the medium 23 is also made from a material through which electromagnetic radiation may transmit, such as an optically diffuse (e.g., translucent) or transparent material. In one particular embodiment, for example, the chromatographic medium 23 is made from an optically diffuse porous membrane formed from materials such as, but not limited to, natural, synthetic, or naturally occurring materials that are synthetically modified, such as polysaccharides (e.g., cellulose materials such as paper and cellulose derivatives, such as cellulose acetate and nitrocellulose); polyether sulfone; polyethylene; nylon; polyvinylidene fluoride (PVDF); polyester; polypropylene; silica; inorganic materials, such as deactivated alumina, diatomaceous earth, MgSO₄, or other inorganic finely divided material uniformly dispersed in a porous polymer matrix, with polymers such as vinyl chloride, vinyl chloride-propylene copolymer, and vinyl chloride-vinyl acetate copolymer; cloth, both naturally occurring (e.g., cotton) and synthetic (e.g., nylon or rayon); porous gels, such as silica gel, agarose, dextran, and gelatin; polymeric films, such as polyacrylamide; and so forth. In one particular embodiment, the chromatographic medium 23 is formed from nitrocellulose and/or polyether sulfone materials. It should be understood that the term “nitrocellulose” refers to nitric acid esters of cellulose, which may be nitrocellulose alone, or a mixed ester of nitric acid and other acids, such as aliphatic carboxylic acids having from 1 to 7 carbon atoms.

The size and shape of the chromatographic medium 23 may generally vary as is readily recognized by those skilled in the art. For instance, a porous membrane strip may have a length of from about 10 to about 100 millimeters, in some embodiments from about 20 to about 80 millimeters, and in some embodiments, from about 40 to about 60 millimeters. The width of the membrane strip may also range from about 0.5 to about 20 millimeters, in some embodiments from about 1 to about 15 millimeters, and in some embodiments, from about 2 to about 10 millimeters. Likewise, the thickness of the membrane strip is generally small enough to allow transmission-based detection. For example, the membrane strip may have a thickness less than about 500 micrometers, in some embodiments less than about 250 micrometers, and in some embodiments, less than about 150 micrometers.

As stated above, the support 21 carries the chromatographic medium 23. For example, the support 21 may be positioned directly adjacent to the chromatographic medium 23 as shown in FIG. 1, or one or more intervening layers may be positioned between the chromatographic medium 23 and the support 21. Regardless, the support 21 may generally be formed from any material able to carry the chromatographic medium 23. Generally, the support 21 is formed from a material that is transmissive to light, such as transparent or optically diffuse (e.g., translucent) materials. Also, it is generally desired that the support 21 is liquid-impermeable so that fluid flowing through the medium 23 does not leak through the support 21. Examples of suitable materials for the support include, but are not limited to, glass; polymeric materials, such as polystyrene, polypropylene, polyester (e.g., Mylar® film), polybutadiene, polyvinylchloride, polyamide, polycarbonate, epoxides, methacrylates, and polymelamine; and so forth. To provide a sufficient structural backing for the chromatographic medium 23, the support 21 is generally selected to have a certain minimum thickness. Likewise, the thickness of the support 21 is typically not so larger as to adversely affect its optical properties. Thus, for example, the support 21 may have a thickness that ranges from about 100 to about 5,000 micrometers, in some embodiments from about 150 to about 2,000 micrometers, and in some embodiments, from about 250 to about 1,000 micrometers. For instance, one suitable membrane strip having a thickness of about 125 micrometers may be obtained from Millipore Corp. of Bedford, Mass. under the name “SHF180UB25.”

As is well known the art, the chromatographic medium 23 may be cast onto the support, wherein the resulting laminate may be die-cut to the desired size and shape. Alternatively, the chromatographic medium 23 may simply be laminated to the support with, for example, an adhesive. In some embodiments, a nitrocellulose or nylon porous membrane is adhered to a Mylar® film. An adhesive is used to bind the porous membrane to the Mylar® film, such as a pressure-sensitive adhesive. Laminate structures of this type are believed to be commercially available from Millipore Corp. of Bedford, Mass. Still other examples of suitable laminate assay device structures are described in U.S. Pat. No. 5,075,077 to Durlev, III, et al., which is incorporated herein in its entirety by reference thereto for all purposes.

The selection of an adhesive for laminating the support 21, the chromatographic medium 23, and/or any other layer of the device may depend on a variety of factors, including the desired optical properties of the detection system and the materials used to form the assay device. For example, in some embodiments, the selected adhesive is optically transparent and compatible with the chromatographic medium 23 and support 21. Optical transparency may minimize any adverse affect that the adhesive might otherwise have on the optical detection system. Suitable optically transparent adhesives may be formed, for instance, from acrylate or (meth)acrylate polymers, such as polymers of (meth)acrylate esters, acrylic or (meth)acrylic acid monomers, and so forth. Exemplary (meth)acrylate ester monomers include monofunctional acrylate or methacrylate esters of non-tertiary alkyl alcohols, such as methyl acrylate, ethyl acrylate, propyl acrylate, n-butyl acrylate, isobutyl acrylate, 2-methylbutyl acrylate, 2-ethylhexyl acrylate, 2-ethylhexyl methacrylate, n-octyl acrylate, n-octyl methacrylate, isooctyl acrylate, isooctyl methacrylate, isononyl acrylate, isodecyl acrylate, isobornyl acrylate, isobornyl methacrylate, vinyl acetate, and mixtures thereof. Exemplary (meth)acrylic acid monomers include acrylic acid, methacrylic acid, beta-carboxyethyl acrylate, itaconic acid, crotonic acid, fumaric acid, and so forth. Several examples of such optically transparent adhesives are described in U.S. Pat. No. 6,759,121 to Alahapperuma, et al., which is incorporated herein in its entirety by reference thereto for all purposes. Further, suitable transparent adhesives may also be obtained from Adhesives Research, Inc. of Glen Rock, Pa. under the name ARclear® 8154, which is an unsupported optically clear acrylic pressure-sensitive adhesive. Other suitable transparent adhesives may be obtained from 3M Corp. of St. Paul, Minn. under the names “9843” or “8146.” In addition, the manner in which the adhesive is applied may also enhance the optical properties of the assay device. For instance, the adhesive may enhance certain optical properties of the support (e.g., diffusiveness). Thus, in one particular embodiment, such an adhesive may be applied in a pattern that corresponds to the areas in which enhanced optical properties are desired.

Referring again to FIG. 1, an absorbent pad 28 is provided on the second surface 14 that generally receives fluid after it migrates through the entire chromatographic medium 23. As is well known in the art, the absorbent pad 28 may also assist in promoting capillary action and fluid flow through the chromatographic medium 23. To initiate the detection of an analyte within the test sample, a user may directly apply the test sample to a portion of the chromatographic medium 23 through which it may then travel in the direction illustrated by arrow “L” in FIG. 1. Alternatively, the test sample may first be applied to a sample pad (not shown) that is in fluid communication with the chromatographic medium 23. Some suitable materials that may be used to form the absorbent pad 28 and/or sample pad include, but are not limited to, nitrocellulose, cellulose, porous polyethylene pads, and glass fiber filter paper. If desired, the sample pad may also contain one or more assay pretreatment reagents, either diffusively or non-diffusively attached thereto.

In the illustrated embodiment, the test sample travels from the sample pad (not shown) to a conjugate pad 22 that is placed in communication with one end of the sample pad. The conjugate pad 22 is formed from a material through which a fluid is capable of passing. For example, in one embodiment, the conjugate pad 22 is formed from glass fibers. Although only one conjugate pad 22 is shown, it should be understood that other conjugate pads may also be used in the present invention.

To facilitate accurate detection of the presence or absence of an analyte within the test sample, a predetermined amount of detection probes may applied at one or more locations of the assay device 20, such as to the conjugate pad 22. Such detection probes contain a luminescent compound that produces an optically detectable signal, such as molecules, polymers, dendrimers, and so forth. For example, suitable fluorescent molecules may include, but not limited to, fluorescein, europium chelates, phycobiliprotein, rhodamine, and their derivatives and analogs. Other suitable fluorescent compounds are semiconductor nanocrystals commonly referred to as “quantum dots.” For example, such nanocrystals may contain a core of the formula CdX, wherein X is Se, Te, S, and so forth. The nanocrystals may also be passivated with an overlying shell of the formula YZ, wherein Y is Cd or Zn, and Z is S or Se. Other examples of suitable semiconductor nanocrystals may also be described in U.S. Pat. No. 6,261,779 to Barbera-Guillem, et al. and U.S. Pat. No. 6,585,939 to Dapprich, which are incorporated herein in their entirety by reference thereto for all purposes.

Further, suitable phosphorescent compounds may include metal complexes of one or more metals, such as ruthenium, osmium, rhenium, iridium, rhodium, platinum, indium, palladium, molybdenum, technetium, copper, iron, chromium, tungsten, zinc, and so forth. Especially preferred are ruthenium, rhenium, osmium, platinum, and palladium. The metal complex may contain one or more ligands that facilitate the solubility of the complex in an aqueous or nonaqueous environment. For example, some suitable examples of ligands include, but are not limited to, pyridine; pyrazine; isonicotinamide; imidazole; bipyridine; terpyridine; phenanthroline; dipyridophenazine; porphyrin, porphine, and derivatives thereof. Such ligands may be, for instance, substituted with alkyl, substituted alkyl, aryl, substituted aryl, aralkyl, substituted aralkyl, carboxylate, carboxaldehyde, carboxamide, cyano, amino, hydroxy, imino, hydroxycarbonyl, aminocarbonyl, amidine, guanidinium, ureide, sulfur-containing groups, phosphorus containing groups, and the carboxylate ester of N-hydroxy-succinimide.

Porphyrins and porphine metal complexes possess pyrrole groups coupled together with methylene bridges to form cyclic structures with metal chelating inner cavities. Many of these molecules exhibit strong phosphorescence properties at room temperature in suitable solvents (e.g., water) and an oxygen-free environment. Some suitable porphyrin complexes that are capable of exhibiting phosphorescent properties include, but are not limited to, platinum (II) coproporphyrin-I and II, palladium (II) coproporphyrin, ruthenium coproporphyrin, zinc(II)-coproporphyrin-I, derivatives thereof, and so forth. Similarly, some suitable porphine complexes that are capable of exhibiting phosphorescent properties include, but not limited to, platinum(II) tetra-meso-fluorophenylporphine and palladium(II) tetra-meso-fluorophenylporphine. Still other suitable porphyrin and/or porphine complexes are described in U.S. Pat. No. 4,614,723 to Schmidt, et al.; U.S. Pat. No. 5,464,741 to Hendrix; U.S. Pat. No. 5,518,883 to Soini; U.S. Pat. No. 5,922,537 to Ewart, et al.; U.S. Pat. No. 6,004,530 to Sagner, et al.; and U.S. Pat. No. 6,582,930 to Ponomarev, et al., which are incorporated herein in their entirety by reference thereto for all purposes.

Bipyridine metal complexes may also be utilized as phosphorescent compounds. Some examples of suitable bipyridine complexes include, but are note limited to, bis[(4,4′-carbomethoxy)-2,2′-bipyridine] 2-[3-(4-methyl-2,2′-bipyridine-4-yl)propyl]-1,3-dioxolane ruthenium (II); bis(2,2′bipyridine)[4-(butan-1-al)-4′-methyl-2,2′-bi-pyridine]ruthenium (II); bis(2,2′-bipyridine)[4-(4′-methyl-2,2′-bipyridine-4′-yl)-butyric acid]ruthenium (II); tris(2,2′bipyridine)ruthenium (II); (2,2′-bipyridine) [bis-bis(1,2-diphenylphosphino)ethylene] 2-[3-(4-methyl-2,2′-bipyridine-4′-yl)propyl]-1,3-dioxolane osmium (II); bis(2,2′-bipyridine)[4-(4′-methyl-2,2′-bipyridine)-butylamine]ruthenium (II); bis(2,2′-bipyridine)[1-bromo-4(4′-methyl-2,2′-bipyridine-4-yl)butane]ruthenium (II); bis(2,2′-bipyridine)maleimidohexanoic acid, 4-methyl-2,2′-bipyridine-4′-butylamide ruthenium (II), and so forth. Still other suitable metal complexes that may exhibit phosphorescent properties may be described in U.S. Pat. No. 6,613,583 to Richter, et al.; U.S. Pat. No. 6,468,741 to Massey, et al.; U.S. Pat. No. 6,444,423 to Meade, et al.; U.S. Pat. No. 6,362,011 to Massey, et al.; U.S. Pat. No. 5,731,147 to Bard, et al.; and U.S. Pat. No. 5,591,581 to Massey, et al., which are incorporated herein in their entirety by reference thereto for all purposes.

Regardless of the type of phosphorescent label utilized, the exposure of the label to quenchers, such as oxygen or water, may result in a disruption of the phosphorescent signal. Thus, to ensure that the phosphorescent labels are capable of emitting the desired signal intensity, they are generally encapsulated within a matrix that acts as a barrier to the relevant quencher. For instance, in some embodiments, the matrix may have a low solubility in water and oxygen, and also be relatively impermeable to water and oxygen. In this manner, the phosphorescent label may be protected from emission decay that would otherwise result from exposure to oxygen or water. For example, the matrix may protect the label such that less than about 30%, in some embodiments less than about 20%, and in some embodiments, less than about 10% of the total phosphorescent signal is quenched when the detection probes are exposed to a particular quencher.

Various types of barrier matrices may be employed in the present invention to inhibit quenching of the phosphorescent compounds. For example, in some embodiments, the phosphorescent compound may be encapsulated within a particle. Some suitable particles that may be suitable for this purpose include, but not limited to, metal oxides (e.g., silica, alumina, etc.), polymer particles, and so forth. For example, latex polymer particles may be utilized, such as those formed from polystyrene, butadiene styrenes, styrene-acrylic-vinyl terpolymer, polymethylmethacrylate, polyethylmethacrylate, styrene-maleic anhydride copolymer, polyvinyl acetate, polyvinylpyridine, polydivinylbenzene, polybutyleneterephthalate, acrylonitrile, vinylchloride-acrylates, derivatives thereof, etc. Other suitable particles may be described in U.S. Pat. No. 5,670,381 to Jou. et al. and U.S. Pat. No. 5,252,459 to Tarcha, et al., which are incorporated herein in their entirety by reference thereto for all purposes.

The phosphorescent compound may be encapsulated within the particulate matrix during and/or after particle formation. In one embodiment, encapsulated latex particles are formed through well-known precipitation techniques. For example, polymer particles may be co-dissolved with the phosphorescent compound in an organic solvent. Thereafter, another solvent may then be added to co-precipitate both the phosphorescent molecules and polymer particles. Some examples of suitable solvents that may be used in such a co-precipitation process include, but are not limited to, water, acetone, acetonitrile, tetrahydrofuran, methylene chloride, cyclohexane, chloroform, ethyl ether, propyl ether, methyl acetate, methyl alcohol, ethyl alcohol, propyl alcohol, pentane, pentene, hexane, methyl ethyl ketone, and other similar solvents.

Besides precipitation, other techniques for forming encapsulated phosphorescent particles may also be used in the present invention. In one embodiment, for example, latex-based phosphorescent particles are formed using swelling techniques. Specifically, a polymer particle is swelled with a swelling agent containing one or more volatile components and phosphorescent molecules. When swollen, the phosphorescent compound may permeate through the polymer particles and become encapsulated therein. Removal of the swelling solvent results in the encapsulated particles. Emulsion polymerization may also be used to form phosphorescent particles. For example, monomers covalently tagged with a phosphorescent moiety may be co-polymerized with other monomers to form phosphorescent particles.

As will be described below, “time-resolved” luminescent detection techniques may be utilized in some embodiments of the present invention. Time-resolved detection involves exciting a luminescent probe with one or more short pulses of light, then typically waiting a certain time after excitation before measuring the remaining luminescent signal, such as from about 1 to about 200 microseconds, and particularly from about 10 to about 50 microseconds. In this manner, any short-lived phosphorescent or fluorescent background signals and scattered excitation radiation are eliminated. This ability to eliminate much of the background signals may result in sensitivities that are 2 to 4 orders greater than conventional fluorescence or phosphorescence. Thus, time-resolved detection is designed to reduce background signals from the illumination source or from scattering processes (resulting from scattering of the excitation radiation) by taking advantage of the characteristics of certain luminescent materials.

To function effectively, time-resolved techniques generally require a relatively long emission lifetime for the luminescent compounds. This is desired so that the compound emits its signal well after any short-lived background signals dissipate. Furthermore, a long luminescence lifetime makes it possible to use low-cost circuitry for time-gated measurements. For example, the detectable compounds may have a luminescence lifetime of greater than about 1 microsecond, in some embodiments greater than about 10 microseconds, in some embodiments greater than about 50 microseconds, and in some embodiments, from about 100 microseconds to about 1000 microseconds. In addition, the compound may also have a relatively large “Stokes shift.” The term “Stokes shift” is generally defined as the displacement of spectral lines or bands of luminescent radiation to a longer emission wavelength than the excitation lines or bands. A relatively large Stokes shift allows the excitation wavelength of a luminescent compound to remain far apart from its emission wavelengths and is desirable because a large difference between excitation and emission wavelengths makes it easier to eliminate the reflected excitation radiation from the emitted signal. Further, a large Stokes shift also minimizes interference from luminescent molecules in the sample and/or light scattering due to proteins or colloids, which are present with some body fluids (e.g., blood). In addition, a large Stokes shift also minimizes the requirement for expensive, high-precision filters to eliminate background interference. For example, in some embodiments, the luminescent compounds have a Stokes shift of greater than about 50 nanometers, in some embodiments greater than about 100 nanometers, and in some embodiments, from about 100 to about 350 nanometers.

For example, one suitable type of fluorescent compound for use in time-resolved detection techniques includes lanthanide chelates of samarium (Sm(III)), dysprosium (Dy(III)), europium (Eu(III)), and terbium (Tb(III)). Such chelates may exhibit strongly red-shifted, narrow-band, long-lived emission after excitation of the chelate at substantially shorter wavelengths. Typically, the chelate possesses a strong ultraviolet excitation band due to a chromophore located close to the lanthanide in the molecule. Subsequent to excitation by the chromophore, the excitation energy may be transferred from the excited chromophore to the lanthanide. This is followed by a fluorescence emission characteristic of the lanthanide. Europium chelates, for instance, have exceptionally large Stokes shifts of about 250 to about 350 nanometers, as compared to only about 28 nanometers for fluorescein. Also, the fluorescence of europium chelates is long-lived, with lifetimes of about 100 to about 1000 microseconds, as compared to about 1 to about 100 nanoseconds for other fluorescent labels. In addition, these chelates have a narrow emission spectra, typically having bandwidths less than about 10 nanometers at about 50% emission. One suitable europium chelate is N-(p-isothiocyanatobenzyl)-diethylene triamine tetraacetic acid-Eu⁺³.

In addition, lanthanide chelates that are inert, stable, and intrinsically fluorescent in aqueous solutions or suspensions may also be used in the present invention to negate the need for micelle-forming reagents, which are often used to protect chelates having limited solubility and quenching problems in aqueous solutions or suspensions. One example of such a chelate is 4-[2-(4-isothiocyanatophenyl)ethynyl]-2,6-bis([N,N-bis(carboxymethyl)amino]methyl)-pyridine [Ref: Lovgren, T., et al.; Clin. Chem. 42, 1196-1201 (1996)]. Several lanthanide chelates also show exceptionally high signal-to-noise ratios. For example, one such chelate is a tetradentate β-diketonate-europium chelate [Ref: Yuan, J. and Matsumoto, K.; Anal. Chem. 70, 596-601 (1998)]. In addition to the fluorescent labels described above, other labels that are suitable for use in the present invention may be described in U.S. Pat. No. 6,030,840 to Mullinax, et al.; U.S. Pat. No. 5,585,279 to Davidson; U.S. Pat. No. 5,573,909 to Singer, et al.; U.S. Pat. No. 6,242,268 to Wieder, et al.; and U.S. Pat. No. 5,637,509 to Hemmila, et al., which are incorporated herein in their entirety by reference thereto for all purposes.

In addition, particularly suitable phosphorescent compounds for time-solved applications may include, platinum (II) coproporhpyrin-I and particles encapsulated with such compounds have an emission lifetime of approximately 50 microseconds, palladium (II) coproporphyrin and particles encapsulated with such compounds have an emission lifetime of approximately 500 microseconds, and ruthenium bipyridyl complexes and particles encapsulated with such compounds have an emission lifetime of from about 1 to about 10 microseconds. Likewise, platinum (II) coproporhpyrin-l has a Stokes shift of approximately 260 nanometers, palladium (II) coproporphyrin has a Stokes shift of approximately 270 nanometers, and ruthenium coproporphyrin has a Stokes shift of approximately 150 nanometers.

Luminescent compounds, such as described above, may be used alone or in conjunction with a particle (sometimes referred to as “beads”). For instance, naturally occurring particles, such as nuclei, mycoplasma, plasmids, plastids, mammalian cells (e.g., erythrocyte ghosts), unicellular microorganisms (e.g., bacteria), polysaccharides (e.g., agarose), etc., may be used. Further, synthetic particles may also be utilized. For example, in one embodiment, latex particles that are labeled with a fluorescent dye are utilized. Although any synthetic particle may be used in the present invention, the particles are typically formed from polystyrene, butadiene styrenes, styreneacrylic-vinyl terpolymer, polymethylmethacrylate, polyethylmethacrylate, styrene-maleic anhydride copolymer, polyvinyl acetate, polyvinylpyridine, polydivinylbenzene, polybutyleneterephthalate, acrylonitrile, vinylchloride-acrylates, and so forth, or an aldehyde, carboxyl, amino, hydroxyl, or hydrazide derivative thereof. Other suitable particles may be described in U.S. Pat. No. 5,670,381 to Jou, et al. and U.S. Pat. No. 5,252,459 to Tarcha, et al. Commercially available examples of suitable fluorescent particles include fluorescent carboxylated microspheres sold by Molecular Probes, Inc. under the trade names “FluoSphere” (Red 580/605) and “TransfluoSphere” (543/620), as well as “Texas Red” and 5- and 6-carboxytetramethylrhodamine, which are also sold by Molecular Probes, Inc.

When utilized, the shape of the particles may generally vary. In one particular embodiment, for instance, the particles are spherical in shape. However, it should be understood that other shapes are also contemplated by the present invention, such as plates, rods, discs, bars, tubes, irregular shapes, etc. In addition, the size of the particles may also vary. For instance, the average size (e.g., diameter) of the particles may range from about 0.1 nanometers to about 1,000 microns, in some embodiments, from about 0.1 nanometers to about 100 microns, and in some embodiments, from about 1 nanometer to about 10 microns. For instance, “micron-scale” particles are often desired. When utilized, such “micron-scale” particles may have an average size of from about 1 micron to about 1,000 microns, in some embodiments from about 1 micron to about 100 microns, and in some embodiments, from about 1 micron to about 10 microns. Likewise, “nano-scale” particles may also be utilized. Such “nano-scale” particles may have an average size of from about 0.1 to about 10 nanometers, in some embodiments from about 0.1 to about 5 nanometers, and in some embodiments, from about 1 to about 5 nanometers.

In some instances, it may be desired to modify the detection probes in some manner so that they are more readily able to bind to the analyte or other substances. In such instances, the detection probes may be modified with certain specific binding members that are adhered thereto to form conjugated probes. Specific binding members generally refer to a member of a specific binding pair, i.e., two different molecules where one of the molecules chemically and/or physically binds to the second molecule. For instance, immunoreactive specific binding members may include antigens, haptens, aptamers, antibodies (primary or secondary), and complexes thereof, including those formed by recombinant DNA methods or peptide synthesis. An antibody may be a monoclonal or polyclonal antibody, a recombinant protein or a mixture(s) or fragment(s) thereof, as well as a mixture of an antibody and other specific binding members. The details of the preparation of such antibodies and their suitability for use as specific binding members are well known to those skilled in the art. Other common specific binding pairs include but are not limited to, biotin and avidin (or derivatives thereof), biotin and streptavidin, carbohydrates and lectins, complementary nucleotide sequences (including probe and capture nucleic acid sequences used in DNA hybridization assays to detect a target nucleic acid sequence), complementary peptide sequences including those formed by recombinant methods, effector and receptor molecules, hormone and hormone binding protein, enzyme cofactors and enzymes, enzyme inhibitors and enzymes, and so forth. Furthermore, specific binding pairs may include members that are analogs of the original specific binding member. For example, a derivative or fragment of the analyte, i.e., an analyte-analog, may be used so long as it has at least one epitope in common with the analyte.

The specific binding members may generally be attached to the detection probes using any of a variety of well-known techniques. For instance, covalent attachment of the specific binding members to the detection probes (e.g., particles) may be accomplished using carboxylic, amino, aldehyde, bromoacetyl, iodoacetyl, thiol, epoxy and other reactive or linking functional groups, as well as residual free radicals and radical cations, through which a protein coupling reaction may be accomplished. A surface functional group may also be incorporated as a functionalized co-monomer because the surface of the detection probe may contain a relatively high surface concentration of polar groups. In addition, although detection probes are often functionalized after synthesis, such as with poly(thiophenol), the detection probes may be capable of direct covalent linking with a protein without the need for further modification. For example, in one embodiment, the first step of conjugation is activation of carboxylic groups on the probe surface using carbodiimide. In the second step, the activated carboxylic acid groups are reacted with an amino group of an antibody to form an amide bond. The activation and/or antibody coupling may occur in a buffer, such as phosphate-buffered saline (PBS) (e.g., pH of 7.2) or 2-(N-morpholino)ethane sulfonic acid (MES) (e.g., pH of 5.3). The resulting detection probes may then be contacted with ethanolamine, for instance, to block any remaining activated sites. Overall, this process forms a conjugated detection probe, where the antibody is covalently attached to the probe. Besides covalent bonding, other attachment techniques, such as physical adsorption, may also be utilized in the present invention.

Referring again to FIG. 1, the chromatographic medium 23 also defines a detection zone 31 within which is immobilized a receptive material that is capable of binding to the conjugated detection probes. For example, in some embodiments, the receptive material may be a biological receptive material. Such biological receptive materials are well known in the art and may include, but are not limited to, antigens, haptens, protein A or G, neutravidin, avidin, streptavidin, captavidin, primary or secondary antibodies (e.g., polyclonal, monoclonal, etc.), and complexes thereof. In many cases, it is desired that these biological receptive materials are capable of binding to a specific binding member (e.g., antibody) present on the detection probes. The receptive material serves as a stationary binding site for complexes formed between the analyte and conjugated detection probes. Specifically, analytes, such as antibodies, antigens, etc., typically have two or more binding sites (e.g., epitopes). Upon reaching the detection zone 31, one of these binding sites is occupied by the specific binding member of the conjugated probe. However, the free binding site of the analyte may bind to the immobilized receptive material. Upon being bound to the immobilized receptive material, the complexed probes form a new ternary sandwich complex.

The detection zone 31 may generally provide any number of distinct detection regions so that a user may better determine the concentration of a particular analyte within a test sample. Each region may contain the same receptive materials, or may contain different receptive materials for capturing multiple analytes. For example, the detection zone 31 may include two or more distinct detection regions (e.g., lines, dots, etc.). The detection regions may be disposed in the form of lines in a direction that is substantially perpendicular to the flow of the test sample through the assay device 20. Likewise, in some embodiments, the detection regions may be disposed in the form of lines in a direction that is substantially parallel to the flow of the test sample through the assay device 20.

Although the detection zone 31 provides accurate results for detecting an analyte, it is sometimes difficult to determine the relative concentration of the analyte within the test sample under actual test conditions. Thus, the assay device 20 may also include a calibration zone 32. In this embodiment, the calibration zone 32 is positioned downstream from the detection zone 31. Alternatively, however, the calibration zone 32 may also be positioned upstream from the detection zone 31. The calibration zone 32 may be provided with a receptive material that is capable of binding to calibration probes or uncomplexed detection probes that pass through the length of the chromatographic medium 23. When utilized, the calibration probes may be formed from the same or different materials as the detection probes. Generally speaking, the calibration probes are selected in such a manner that they do not bind to the receptive material at the detection zone 31.

The receptive material of the calibration zone 32 may be the same or different than the receptive material used in the detection zone 31. For example, in one embodiment, the receptive material is a biological receptive material. In addition, it may also be desired to utilize various non-biological materials for the receptive material of the calibration zone 32. The polyelectrolytes may have a net positive or negative charge, as well as a net charge that is generally neutral. For instance, some suitable examples of polyelectrolytes having a net positive charge include, but are not limited to, polylysine (commercially available from Sigma-Aldrich Chemical Co., Inc. of St. Louis, Mo.), polyethylenimine; epichlorohydrin-functionalized polyamines and/or polyamidoamines, such as poly(dimethylamine-co-epichlorohydrin); polydiallyldimethyl-ammonium chloride; cationic cellulose derivatives, such as cellulose copolymers or cellulose derivatives grafted with a quaternary ammonium water-soluble monomer; and so forth. In one particular embodiment, CelQuat® SC-230M or H-100 (available from National Starch & Chemical, Inc.), which are cellulosic derivatives containing a quaternary ammonium water-soluble monomer, may be utilized. Moreover, some suitable examples of polyelectrolytes having a net negative charge include, but are not limited to, polyacrylic acids, such as poly(ethylene-co-methacrylic acid, sodium salt), and so forth. It should also be understood that other polyelectrolytes may also be utilized in the present invention, such as amphiphilic polyelectrolytes (i.e., having polar and non-polar portions). For instance, some examples of suitable amphiphilic polyelectrolytes include, but are not limited to, poly(styryl-b-N-methyl 2-vinyl pyridinium iodide) and poly(styryl-b-acrylic acid), both of which are available from Polymer Source, Inc. of Dorval, Canada. Further examples of internal calibration systems that utilize polyelectrolytes are described in more detail in U.S. patent app. Publication No. 2003/0124739 to Song, et al., which is incorporated herein in it entirety by reference thereto for all purposes.

In some cases, the chromatographic medium 23 may also define a control zone (not shown) that gives a signal to the user that the assay is performing properly. For instance, the control zone (not shown) may contain an immobilized receptive material that is generally capable of forming a chemical and/or physical bond with probes or with the receptive material immobilized on the probes. Some examples of such receptive materials include, but are not limited to, antigens, haptens, antibodies, protein A or G, avidin, streptavidin, secondary antibodies, and complexes thereof. In addition, it may also be desired to utilize various non-biological materials for the control zone receptive material. For instance, in some embodiments, the control zone receptive material may also include a polyelectrolyte, such as described above, that may bind to uncaptured probes. Because the receptive material at the control zone is only specific for probes, a signal forms regardless of whether the analyte is present. The control zone may be positioned at any location along the medium 23, but is typically positioned upstream from the detection zone 31.

Various formats may be used to test for the presence or absence of an analyte using the assay device 20. For instance, a “sandwich” format typically involves mixing the test sample with detection probes conjugated with a specific binding member (e.g., antibody) for the analyte to form complexes between the analyte and the conjugated probes. These complexes are then allowed to contact a receptive material (e.g., antibodies) immobilized within the detection zone. Binding occurs between the analyte/probe conjugate complexes and the immobilized receptive material, thereby localizing “sandwich” complexes that are detectable to indicate the presence of the analyte. This technique may be used to obtain quantitative or semi-quantitative results. Some examples of such sandwich-type assays are described by U.S. Pat. No. 4,168,146 to Grubb, et al. and U.S. Pat. No. 4,366,241 to Tom, et al., which are incorporated herein in their entirety by reference thereto for all purposes. In a competitive assay, the labeled probe is generally conjugated with a molecule that is identical to, or an analog of, the analyte. Thus, the labeled probe competes with the analyte of interest for the available receptive material. Competitive assays are typically used for detection of analytes such as haptens, each hapten being monovalent and capable of binding only one antibody molecule. Examples of competitive immunoassay devices are described in U.S. Pat. No. 4,235,601 to Deutsch, et al., U.S. Pat. No. 4,442,204 to Liotta, and U.S. Pat. No. 5,208,535 to Buechler. et al., which are incorporated herein in their entirety by reference thereto for all purposes. Various other device configurations and/or assay formats are also described in U.S. Pat. No. 5,395,754 to Lambotte, et al.; U.S. Pat. No. 5,670,381 to Jou. et al.; and U.S. Pat. No. 6,194,220 to Malick. et al., which are incorporated herein in their entirety by reference thereto for all purposes.

II. Luminescent Detection System

Regardless of the particular type of assay device utilized, a luminescent 25 detection system is employed in accordance with the present invention to detect the presence or absence of an analyte. The luminescent system utilized in the present invention employs transmission-based photometric detection techniques to minimize signal interference and to reduce the need for expensive and complex instruments. Referring again to FIG. 1, for example, the detection system is schematically illustrated and employs a luminescent reader 50 that contains an illumination source 52 and a detector 54. As shown, the detector 54 is positioned adjacent to the support 21 and the illumination source 52 is positioned adjacent to the second surface 14 of the chromatographic medium 23. Likewise, the detector 54 may be positioned adjacent to the second surface 14 of the chromatographic medium 23 and the illumination source 52 may be positioned adjacent to the support 21. Thus, the illumination source 52 may emit light simultaneously onto the detection and calibration zones 31 and 32, and the detector 54 may likewise also simultaneously receive a luminescent signal from the excited probes at the detection and calibration zones 31 and 32. Alternatively, the illumination source 52 may be constructed to successively emit light onto the detection zone 31 and the calibration zone 32. In addition, a separate illumination source and/or detector (not shown) may also be used for the calibration zone 32.

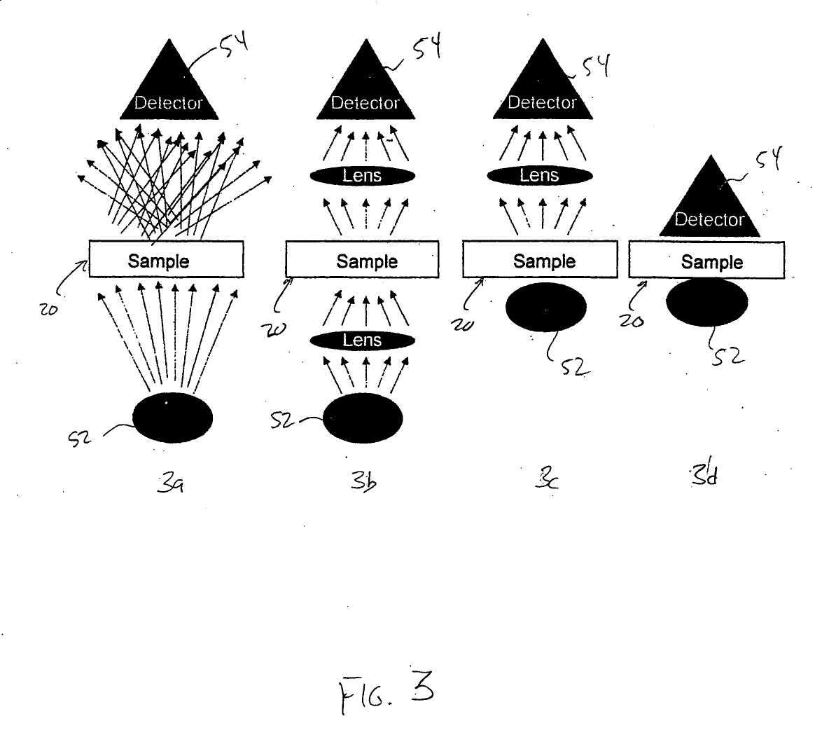

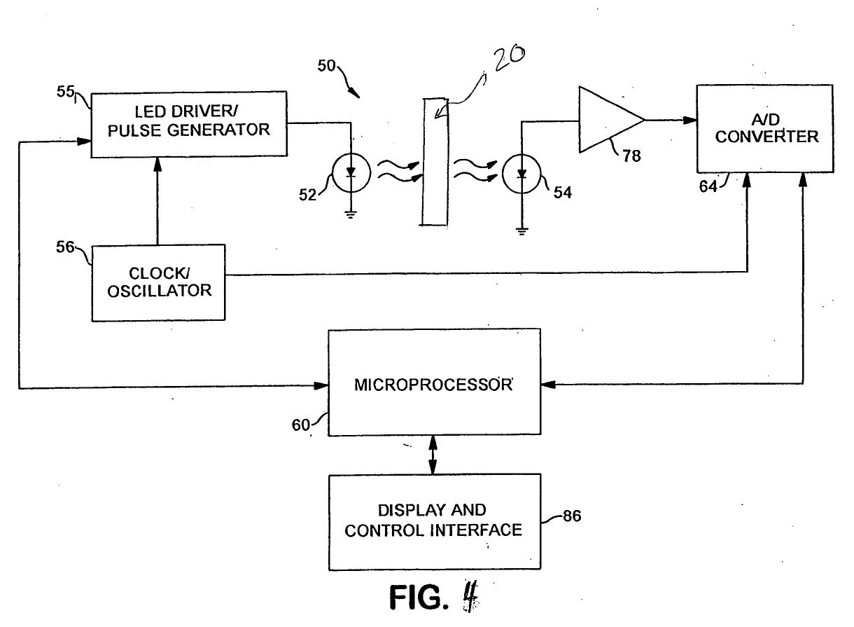

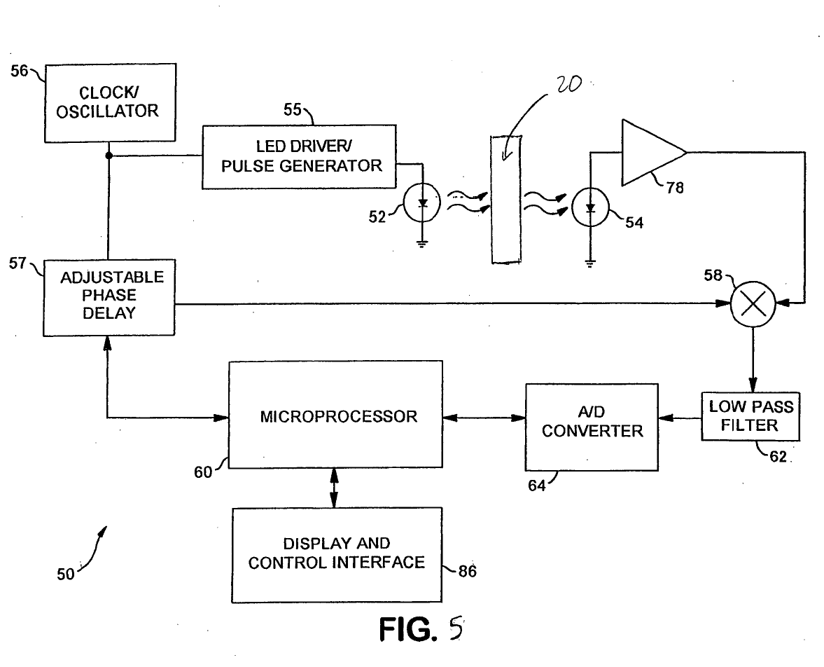

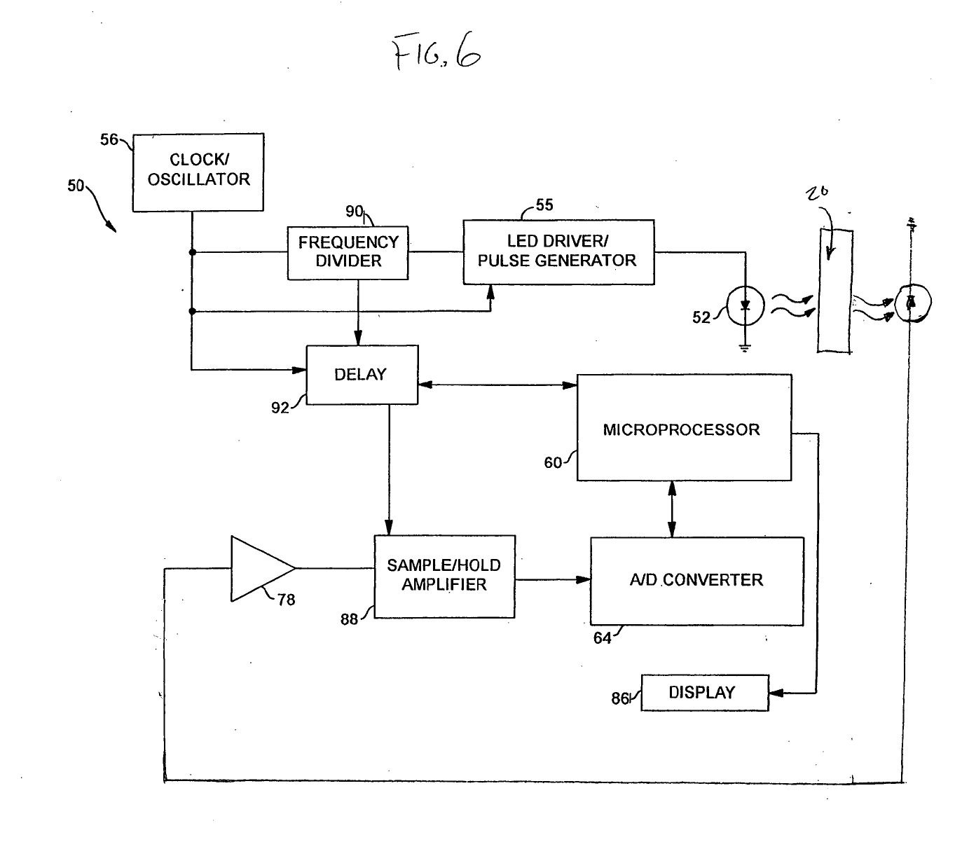

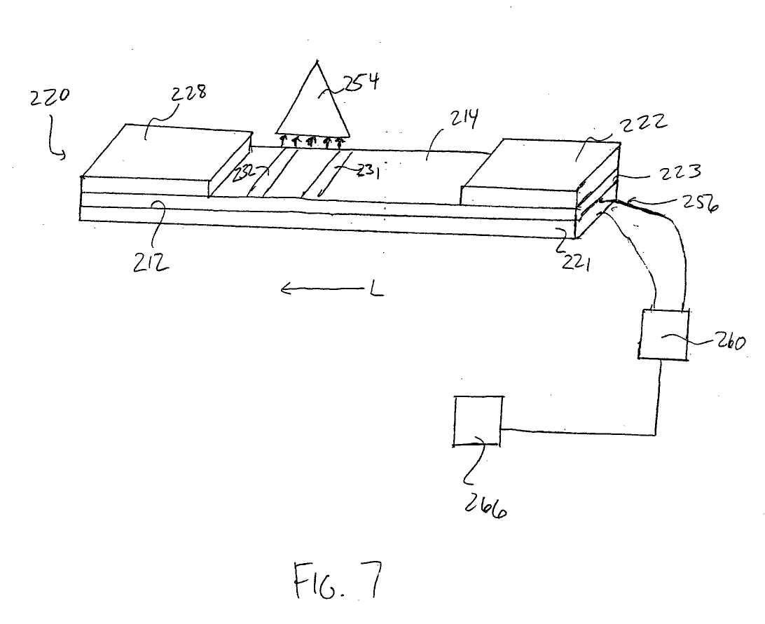

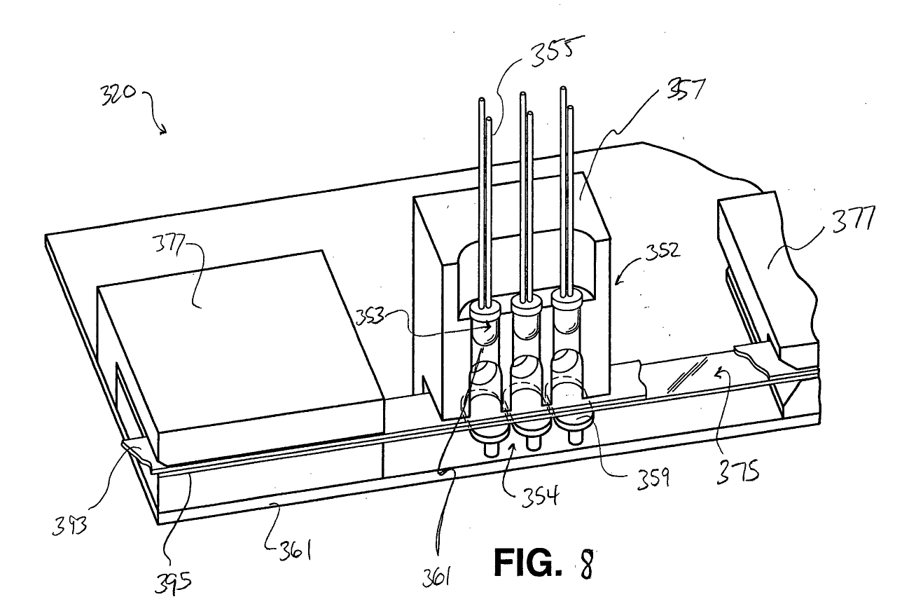

To improve the signal-to-noise ratio of the optical detection system without the need for certain types of complex and expensive optical components, such as lenses or other light guiding elements, the distance of the illumination source 52 and/or detector 54 from the assay device 20 may be minimized in some embodiments. For instance, as shown in FIG. 3a, light (indicated by directional arrows) traveling a relatively large distance tends to diffuse, thereby causing some photons to miss the test sample or the detector 54. To reduce light scattering, lenses may be employed to focus the light in the desired direction, such as shown in FIG. 3b. However, as shown in FIGS. 3c and 3d, the need for such expensive and complex equipment may be reduced by simply moving the illumination source 52 and/or detector 54 closer to the assay device 20. The use of a shorter light path results in less diffusion of the light. For example, FIG. 3c illustrates an embodiment in which the illumination source 52 is positioned closer to the assay device 20, and FIG. 3d illustrates an embodiment in which both the illumination source 52 and detector 54 are positioned closer to the assay device 20. Thus, in some embodiments, the illumination source 52 and/or detector 54 may be positioned less than about 5 millimeters, in some embodiments less than about 3 millimeters, and in some embodiments, less than about 2 millimeters from the assay device 20. As will be discussed in more detail below, the illumination source 52 and/or detector 54 may, in some cases, directly contact the chromatographic medium 23. For example, the illumination source 52 may carry the medium 23, thereby also functioning as its support. In other cases, however, it may be desired to keep the illumination source 52 and/or detector 54 at a distance that is large enough to avoid contamination of any biological reagents. For example, the illumination source 52 and/or detector 54 may sometimes be positioned at a distance of from about 1 to about 3 millimeters from the assay device 20.

Generally speaking, the illumination source 52 may be any device known in the art that is capable of providing electromagnetic radiation at a sufficient intensity to excite luminescent probes. The electromagnetic radiation may include light in the visible or ultraviolet range. For example, suitable illumination sources that may be used in the present invention include, but are not limited to, light emitting diodes (LED), flashlamps, cold-cathode fluorescent lamps, electroluminescent lamps, and so forth. The illumination may be multiplexed and/or collimated. In some cases, the illumination may be pulsed to reduce any background interference. Further, illumination may be continuous or may combine continuous wave (CW) and pulsed illumination where multiple illumination beams are multiplexed (e.g., a pulsed beam is multiplexed with a CW beam), permitting signal discrimination between a signal induced by the CW source and a signal induced by the pulsed source. For example, in some embodiments, LEDs (e.g., aluminum gallium arsenide red diodes, gallium phosphide green diodes, gallium arsenide phosphide green diodes, or indium gallium nitride violet/blue/ultraviolet (UV) diodes) are used as the pulsed illumination source 52. One commercially available example of a suitable UV LED excitation diode suitable for use in the present invention is Model NSHU550E (Nichia Corporation), which emits 750 to 1000 microwatts of optical power at a forward current of 10 milliamps (3.5-3.9 volts) into a beam with a full-width at half maximum of 10 degrees, a peak wavelength of 370-375 nanometers, and a spectral half-width of 12 nanometers.

In some cases, the illumination source 52 may provide diffuse illumination to the assay device 20. In this manner, the reliance on certain external optical components, such as diffusers, may be virtually eliminated. For example, in some embodiments, the resin package containing an LED may be provided with a diffusive surface to achieve diffuse illumination. Alternately, an array of multiple point light sources (e.g., LEDs) may simply be employed to provide relatively diffuse illumination to the device 20. Another particularly desired illumination source that is capable of providing diffuse illumination in a relatively inexpensive manner is an electroluminescent (EL) device. An EL device is generally a capacitor structure that utilizes a luminescent material (e.g., phosphor particles) sandwiched between electrodes, at least one of which is transparent to allow light to escape. Application of a voltage across the electrodes generates a changing electric field within the luminescent material that causes it to emit light.

Any of a variety of known EL devices may be employed as the illumination source 52. For example, EL devices that employ “inorganic” or “organic“luminescent materials may be utilized in the present invention. Suitable “organic” EL devices include low and high molecular weight devices. Likewise, suitable inorganic EL devices include dispersion and thin-film phosphors. Dispersion EL devices generally contain a dispersion of powder luminescent material in a binder, which is sandwiched between electrode layers. On the other hand, thin-film EL devices include a luminescent thin film that is sandwiched between a pair of insulating thin films and a pair of electrode layers, and is disposed on an electrically insulating substrate. Although certainly not required, the dispersion-type EL devices are particularly desired in certain embodiments of the present invention due to their relatively low cost and ease of manufacture.

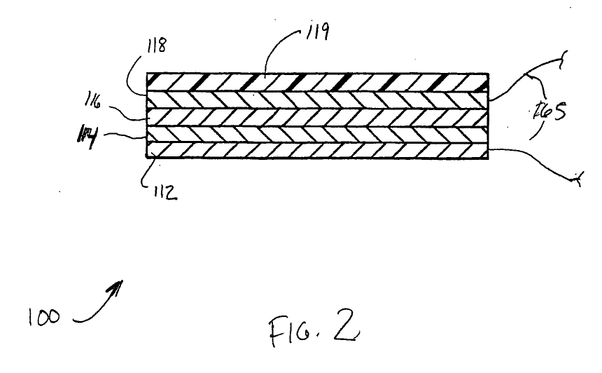

Referring to FIG. 2, for instance, one embodiment of a dispersion-type EL device 100 that may be used in the present invention is illustrated. As shown, the EL device 100 has a cathode 112, a dielectric layer 114, a luminescent layer 116, an anode 118, and a film 119. Additional water-impervious protective layers (not shown) may optionally be applied to the cathode 112 and film 119 if desired. Leads 165 are electrically attached to the respective cathode and anode layers 112 and 118. The cathode 112 may be formed from a metal (including metalloids) or alloys thereof (including intermetallic compounds). Examples of suitable materials for forming the cathode 112 include, but are not limited to, carbon; metals, such as aluminum, gold, silver, copper, platinum, palladium, iridium, and alloys thereof; and so forth. The thickness of the cathode 112 may generally vary, and may be deposited onto an electrically insulating substrate (not shown). The substrate, for instance, may be formed from ceramic materials, such as alumina (Al₂O₃), quartz glass (SiO₂), magnesia (MgO), forsterite (2MgO.SiO₂), steatite (MgO.SiO₂), mullite (3Al₂O₃.2SiO₂), beryllia (BeO), zirconia (ZrO₂), aluminum nitride (AlN), silicon nitride (SiN), silicon carbide (SiC), glass, heat resistant glass, and so forth. In addition, polymeric materials may also be used to form the substrate, such as, polypropylene, polyethylene terephthalate, polyvinyl chloride, polymethylmethacrylate, and so forth.

The dielectric layer 114 is disposed on the cathode 112. The material of which the dielectric layer 114 is formed may generally vary as is well known to those skilled in the art. For example, suitable materials include, but are not limited to, perovskite structure dielectric and ferroelectric materials, such as BaTiO₃, (BaₓCa_{1-x )TiO}₃, (BaₓSr₁₋ₓ)TiO₃, PbTiO₃ and Pb(ZrₓTi₁₋ₓ)O₃ (known as “PZT”); complex perovskite relaxation type ferroelectric materials, such as Pb(Mg_{1/3}Nb_{2/3})O₃; bismuth layer compounds, such as Bi₄Ti₃O₁₂ and SrBi₂Ta₂O₉; and tungsten bronze type ferroelectric materials, such as (SrₓBa₁₋ₓ)Nb₂O₆ and PbNb₂O₆. Still other suitable dielectric materials for use in the dielectric layer 114 may include dielectric material, such as SiO₂, SiN, SiON, ZrO₂, Al₂O₃, Al₃N₄, Y₂O₃, Ta₂O₅, and so forth. In one particular embodiment, the dielectric layer 114 is formed from barium titanate (BaTiO₃).

The dielectric layer 114 may be formed using any of a variety of techniques known to those skilled in the art. For example, the dielectric material used to form the layer 114 may first be admixed with a suitable solvent. Such solvents may include, for instance, glycol ethers, alkyl ketones and aromatic solvents. Suitable glycol ethers may include propylene glycol methyl ether, dipropylene glycol methyl ether, tripropylene glycol methyl ether, ethylene glycol ethyl ether, diethylene glycol butyl ether, and so forth. Suitable alkyl ketones may include lower alkyl ketones, such as acetone, methyl ethyl ketone, ethyl ketone and methylisobutyl ketone, and so forth. Suitable aromatic solvents may include toluene, xylene, and so forth. In one embodiment, barium titanate is added to a solvent in an amount from about 70% to about 90% by weight. The barium titanate and the solvent are then stirred together to form a homogeneous slurry.

Upon mixing with a solvent, the dielectric material may also be mixed with a binder. For example, in some embodiments, the binder is added in an amount from about 10 to about 30 parts of the slurry. Suitable binders are well known and include, for instance, epoxy resins, polystyrene, polyethylene, polyvinyl butyral, polyvinyl chloride, polyvinyl acetate, polyvinyl alcohol, polyesters, polyamides, polyacrylonitrile, polyacrylate, polymethylmethacrylate and the like. In some embodiments, the binder is an adhesive thermoplastic reaction product of phenols and an excess of an epihalohydrin. Suitable phenols include bisphenol A, dichlorobisphenol A, tetrachlorobisphenol A, tetrabromobisphenol A, bisphenol F and bisphenol ACP. The reaction is carried out in the presence of a glycol ether or other suitable solvent. To this reaction product is added a resin such as a urethane or an epoxy resin in the range of from about 5 to 6 parts of resin to about 1 part of the epihalohydrin/phenol reaction product. Such binders are described in more detail in U.S. Pat. No. 4,560,902 to Kardon and U.S. Pat. No. 5,352,951 to Kardon, et al., which are incorporated herein in their entirety by reference thereto for all purposes.

If desired, water may be added to the binder system at this step or following assembly of the EL device 100. The water may be stirred into the slurry before or after removal of the solvent. The amount of water added to the binder will vary somewhat in accordance with the amount of water the particular binder employed can absorb. For instance, at least about 1 part per million (“ppm”) (0.0001%) of water may be present and up to the maximum amount of water the binder will absorb. Cyanoethyl polyvinyl alcohol binders, for example, typically absorb a maximum of about 40,000 ppm (4.0%) of water. Cyanoalkylated pullulan binders, on the other hand, typically absorb a maximum of about 100,000 ppm (10.0%) of water. However, in most cases, the amount of water added to the binder is from about 500 ppm (0.05%) to about 20,000 ppm (2.0%). The thickness of the resultant barium titanate/resin binder layer 114 is typically from about 0.2 to about 6 mils.

Referring again to FIG. 2, the EL device 100 also includes a luminescent layer 116 disposed on the dielectric layer 114. The material of which the luminescent layer 116 may include phosphor particles. Suitable phosphor particles may include a variety of metal oxide, sulfide, fluoride, and silicate compounds. For example, such phosphor particles may include manganese- and arsenic-activated zinc silicate (P39 phosphor), titanium-activated zinc silicate, manganese-activated zinc silicate (P1 phosphor), cerium-activated yttrium silicate (P47 phosphor), manganese-activated magnesium silicate (P13 phosphor), lead- and manganese-activated calcium silicate (P25 phosphor), terbium-activated yttrium silicate, terbium-activated yttrium oxide, terbium-activated yttrium aluminum oxide, terbium-activated gadolinium oxide, terbium-activated yttrium aluminum gallium oxide, europium-activated yttrium oxide, europium-activated yttrium vanadium oxide, europium-activated yttrium oxysulfide, manganese-activated zinc sulfide, cesium-activated strontium sulfide, thulium-activated zinc sulfide, samarium-activated zinc sulfide, europium-activated calcium sulfide, terbium-activated zinc-sulfide, and cesium-activated calcium sulfide, and so forth.

The color emitted by the phosphor particles can be defined during the manufacture of the phosphor or by blending phosphors of different colors to achieve composite color. Some specific examples of suitable phosphors include manganese-activated zinc sulfide (yellowish orange light emission), cesium-activated strontium sulfide (blue light emission), thulium-activated zinc sulfide (blue light emission), samarium-activated zinc sulfide (red light emission), europium-activated calcium sulfide (red light emission), terbium-activated zinc-sulfide (green light emission), and cesium-activated calcium sulfide (green light emission).

Phosphor particles typically have an average size of less than about 15 micrometers, in some embodiments less than about 10 micrometers, and in some embodiments, less than about 5 micrometers. The luminescent layer 116 may be formed using any of a variety of techniques known to those skilled in the art. For example, the encapsulated phosphor particles may be admixed with a solvent, such as described above. The amount of phosphor particles added to the solvent may range, for instance, from about 60% to about 95%, and in some embodiments, from about 75% to about 85% by weight of the mixture. Likewise, after mixing, a binder, such as described above, is also mixed with the phosphor particle slurry. The binder is typically present in an amount of from about 5 to about 40 parts. If desired, the phosphor particles may also be encapsulated within a protective material to form a water barrier as is well known in the art. Suitable protective materials for encapsulating the phosphor particles include, for instance, liquid crystals, polymeric binders, ceramic materials (e.g., colloidal silica, alumina, etc.), and so forth. Encapsulation techniques are described in more detail in U.S. Pat. No. 4,097,776 to Allinikov; U.S. Pat. No. 4,513,023 to Warv; U.S. Pat. No. 4,560,902 to Kardon; and U.S. Pat. No. 5,352,951 to Kardon, et al., which are incorporated herein in their entirety by reference thereto for all purposes.

The phosphor particles preferably are deposited in a smooth, homogeneous layer by any of a variety of techniques known to one of skill in the art. Such techniques include settling techniques, slurry methods (such as screen printing, spin coating, and spin casting), electrophoresis, or dusting methods (such as electrostatic dusting, “phototacky” methods, and high pressure dusting). Settling techniques and slurry methods involve forming a dispersion of the phosphor particles in a suitable liquid medium. One particularly desired deposition method is screen printing. A suitable thickness for the phosphor/binder layer 116 when dried is about 0.2 to about 6 mils.

In addition to the layers mentioned above, the EL device 100 also includes an anode 118 formed on a film 119, both of which are disposed over the luminescent layer 116. Desirably, the materials used for the layers 118 and 119 are optically transparent. For example, the anode 118 may be formed from a inorganic conductive oxide, such as indium oxide, indium tin oxide (ITO), tin oxide, and antimony tin oxide. In one embodiment, an indium tin oxide (ITO) layer is utilized that has a thickness of about 0.2 to 1 micrometers. Likewise, a suitable material for use as the film 119 may be a polymer film (e.g., polyester). It should be understood that the embodiments described above are merely exemplary, and that any other known EL device may generally be used in the present invention. For instance, other suitable EL devices are described in U.S. Pat. No. 6,004,686 to Rasmussen, et al.; U.S. Pat. No. 6,432,516 to Terasaki, et al.; U.S. Pat. No. 6,602,618 to Watanabe, et al.; U.S. Pat. No. 6,479,930 to Tanabe, et al.; U.S. Pat. No. 6,723,192 to Nagano, et al.; and U.S. Pat. No. 6,734,469 to Yano, et al., as well as U.S. patent application Publication Nos. 2003/0193289 to Shirakawa, et al.; 2004/0119400 to Takahashi, et al., and 2004/0070195 to Nelson, et al., all of which are incorporated herein in their entirety by reference thereto for all purposes.

When utilized as the illumination source 52 (FIG. 1), EL devices may provide a variety of benefits for the optical detection system. For instance, unlike point light sources used with many conventional optical detection systems (e.g. LEDs), EL devices emit relatively homogeneous and diffuse light, and may thus provide uniform illumination. This may eliminate the need for additional diffusers often required in other point-source illumination systems. In addition, the light intensity emitted by EL device may be easily controlled by simply varying the voltage or the frequency of the drive signal. Thus, an EL device allows for the use of optical readers that are relatively simple, portable, and inexpensive.

In FIG. 1, the illumination source 52 is shown as a component that is separate from the assay device 20. However, the present invention also contemplates embodiments in which the illumination source is integral with the assay device 20. For example, in some embodiments, the support 21 is an EL device that functions simultaneously as a light source for the optical detection system and as a physical carrier for the chromatographic medium 23. The use of an EL device as the support 21 provides a substantial benefit to the resulting optical detection system by eliminating the need for additional light sources, which are often costly and lead to overly complex and space-consuming systems. That is, the EL device may be laminated to the chromatographic medium 23 and simultaneously function as the support 21 and light source for the optical detection system. The EL device may be selected to possess a certain degree of flexibility that allows it to be readily manipulated and/or cut into the desired shape and size for the assay device 20. One commercially available EL device that has enough strength and flexibility for use as the support 21 is a lamp kit available from Graphic Solutions Int'l, LLC of Burr Ridge, Ill. under the name “Proto-Kut.”

One particular embodiment of the present invention in which an EL device is employed as the support for the assay device is shown in FIG. 7. Specifically, an assay device 220 is depicted that includes a chromatographic medium 223, an EL device 221, an absorbent pad 228, and a conjugate pad 222. The medium 223 has a first surface 212 and a second surface 214, wherein the first surface 212 is positioned adjacent to the EL device 221. A detection zone 231 and calibration zone 232 are defined by the medium 223 for providing detection and calibration signals. Further, a detector 254 positioned adjacent to the second surface 214 of the medium 223. In this particular embodiment, the EL device 221 functions as both the illumination source and the support for the medium 223. Leads 256 for the EL device 221 are connected to a driver circuit 260 via wiring, which in turn, is connected to a power source 266. The details of the driver circuit 260 and power source 266 depend on the requirements of the particular EL device. For example, because the EL device 221 may be relatively small due to the corresponding small size of the assay device 220, a low voltage circuit and battery power source may be employed to reduce the cost and complexity of the system. However, higher voltage circuits may also be used, such as a driver circuit that converts DC voltage into an AC output for driving the EL device 221. Such AC inverters may generate around 60 to 300 volts AC at 50 to 5000 Hertz. Driver circuits suitable for this purpose are commercially available.

Referring again to FIG. 1, the detector 54 may generally be any device known in the art that is capable of sensing an optical signal. For instance, the detector 54 may be an electronic imaging detector that is configured for spatial discrimination. Some examples of such electronic imaging sensors include high speed, linear charge-coupled devices (CCD), charge-injection devices (CID), complementary-metal-oxide-semiconductor (CMOS) devices, and so forth. Such image detectors, for instance, are generally two-dimensional arrays of electronic light sensors, although linear imaging detectors (e.g., linear CCD detectors) that include a single line of detector pixels or light sensors, such as, for example, those used for scanning images, may also be used. Each array includes a set of known, unique positions that may be referred to as “addresses.” Each address in an image detector is occupied by a sensor that covers an area (e.g., an area typically shaped as a box or a rectangle). This area is generally referred to as a “pixel” or pixel area. A detector pixel, for instance, may be a CCD, CID, or a CMOS sensor, or any other device or sensor that detects or measures light. The size of detector pixels may vary widely, and may in some cases have a diameter or length as low as 0.2 micrometers.

In other embodiments, the detector 54 may be a light sensor that lacks spatial discrimination capabilities. For instance, examples of such light sensors may include photomultiplier devices, photodiodes, such as avalanche photodiodes or silicon photodiodes, and so forth. Silicon photodiodes are sometimes advantageous in that they are inexpensive, sensitive, capable of high-speed operation (short risetime/high bandwidth), and easily integrated into most other semiconductor technology and monolithic circuitry. In addition, silicon photodiodes are physically small, which enables them to be readily incorporated into a system for use with a membrane-based device. If silicon photodiodes are used, then the wavelength range of the emitted signal may be within their range of sensitivity, which is 400 to 1100 nanometers.

Referring to FIGS. 8 and 9, for example, one embodiment of a luminescent detection system 320 is shown that employs a photodiode-based detector 354 and an LED-based illumination source 352. In this particular embodiment, three LEDs 353 are utilized, although any number of LEDs may generally be employed in the present invention. Each LED 353 is connected to respective leads 355 and enclosed within a reflective housing 357. For purposes of illustration, only a portion of the housing 357 is shown in FIGS. 8 and 9; however, in most embodiments, the housing 357 will be disposed concentrically around the LEDs 353 to restrict the illumination to only the area of interest. The housing 357 defines light cavities 361 in which the LEDs 353 are mounted for providing illumination to an assay strip 375, which includes a membrane 393 and support 395 held in place by two sample holders 377. The detector 354 shown in FIGS. 8 and 9 employs three photodiodes 359 correspond to the number of LEDs 353, although any number of photodiodes 359 may of course be employed in the present invention. The photodiodes 359 are mounted on a base 361 and positioned close to the assay strip 375 in a manner that corresponds to the lateral position of the LEDs 353.

Although it is generally desired to limit the use of external optical components to reduce costs and complexity, such components may nevertheless be utilized in some embodiments of the present invention. If utilized, separate optical components may be used for the illumination source 52 and detector 54, or they may share common optical components. For example, optical filters (not shown) may be disposed adjacent to the illumination source 52 and/or detector 54. The optical filters may have high transmissibility in a desired wavelength range(s) and low transmissibility in one or more undesirable wavelength band(s) to filter out undesirable wavelengths from the illumination source 52. Undesirable wavelength ranges generally include those wavelengths that produce detectable sample autofluoresence and/or are within about 25 to about 100 nanometers of excitation maxima wavelengths and thus are potential sources of background noise from scattered excitation illumination. Several examples of optical filters that may be utilized in the present invention include, but are not limited to, dyed plastic resin or gelatin filters, dichroic filters, thin multi-layer film interference filters, plastic or glass filters, epoxy or cured transparent resin filters. In one embodiment, the detector 54 and/or illumination source 52 may be embedded or encapsulated within the filter.

Optical diffusers may also be utilized in the present invention to scatter light in a certain direction, such as toward and/or away from the detection zone. Optical diffusers are particularly useful in conjunction with a detection system that employs a “point” light source, such as a light-emitting diode (LED). For example, suitable optical diffusers may include diffusers that scatter light in various directions, such as ground glass, opal glass, opaque plastics, chemically etched plastics, machined plastics, and so forth. Opal glass diffusers contain a milky white “opal” coating for evenly diffusing light, thereby producing a near Lambertian source. Other suitable light-scattering diffusers include polymeric materials (e.g., polyesters, polycarbonates, etc.) that contain a light-scattering material, such as titanium dioxide or barium sulfate particles. In other embodiments, holographic diffusers may be utilized that both homogenize and impart predetermined directionality to light rays emanating from the illumination source. Such diffusers may contain a micro-sculpted surface structure that controls the direction in which light propagates. Examples of such holographic diffusers are described in more detail in U.S. Pat. No. 5,534,386 to Petersen, et al., which is incorporated herein in its entirety by reference thereto for all purposes.

In addition, a lens may also be used to collect and focus light. One particular embodiment of the present invention utilizes a micro-lens to focus light toward the test sample and/or detector 54. Suitable micro-optic lenses include, but are not limited to, gradient index (GRIN) lenses, ball lenses, Fresnel lenses, and so forth. For example, a gradient index lens is generally cylindrical, and has a refractive index that changes radially with a parabolic profile. A ball lens is generally spherical, and has a refractive index that is radially constant. Because of their relatively small size, such micro-lenses may be particularly advantageous in the present invention. Any of a variety of well-known techniques may be utilized to form the micro-lens. For example, micro-lenses may be formed by submerging a substrate (e.g., silicon or quartz) into a solution of alkaline salt so that ions are exchanged between the substrate and the salt solution through a mask formed on the substrate, thereby obtaining a substrate having a distribution of indexes of refraction corresponding to the pattern of the mask. In addition, a photosensitive monomer may be irradiated with ultraviolet rays to polymerize an irradiated portion of the photosensitive monomer. Thus, the irradiated portion bulges into a lens configuration under an osmotic pressure occurring between the irradiated portion and the non-irradiated portion. In another embodiment, a photosensitive resin may be patterned into circles, and heated to temperatures above its softening point to enable the peripheral portion of each circular pattern to sag by surface tension. This process is referred to as a “heat sagging process.” Further, a lens substrate may simply be mechanically shaped into a lens. Still other suitable techniques for forming a micro-lens or other micro-optics are described in U.S. Pat. No. 5,225,935 to Watanabe, et al.; U.S. Pat. No. 5,910,940 to Guerra; and U.S. Pat. No. 6,411,439 to Nishikawa, which are incorporated herein in their entirety by reference thereto for all purposes.

Further, a mask, such as a black coating or dye, may be utilized to prevent light from passing through one or more sections of the assay device 20. Light guiding elements may also be utilized to direct light in a desired direction, such as a single optical fiber, fiber bundle, segment of a bifurcated fiber bundle, large diameter light pipe, planar waveguide, attenuated total reflectance crystal, dichroic mirror, plane mirror or other light guiding elements. Still other examples of optically functional materials that may be used in the present invention described in U.S. Pat. No. 5,827,748 to Golden; U.S. Pat. No. 6,084,683 to Bruno, et al.; U.S. Pat. No. 6,235,241 to Catt, et al.; U.S. Pat. No. 6,556,299 to Rushbrooke. et al.; and U.S. Pat. No. 6,566,508 to Bentsen, et al., which are incorporated herein in their entirety by reference thereto for all purposes.

If desired, the optical properties of the assay device itself may be selectively tailored to the optical requirements of the detection system. For example, referring again to FIG. 1, one embodiment of the present invention employs selective control of the support 21 to optimize the performance of the optical detection system. In one particular embodiment, for example, the support 21 is optically transmissive to allow for light to travel from the illumination source 52 to the detector 54. In addition, the support 21 may function as an optical filter of the detection system. Thus, in the illustrated embodiment, light from the illumination source 52 is absorbed by probes (not shown) present at the detection zone 31 and/or calibration zone 32. The probes emit a signal that is attenuated by the optical filter before reaching the detector 54. The optical filter may, for example, have high transmissibility in the emission wavelength range(s) and low transmissibility in one or more undesirable wavelength band(s) to filter out undesirable wavelengths from the detector 54. The optical detection system may also include an additional optical filter (not shown) positioned between the illumination source 52 and the chromatographic medium 23. This additional optical filter may have high transmissibility in the excitation wavelength range(s) and low transmissibility in one or more undesirable wavelength band(s). Alternatively, an additional optical filter may be integrated into the illumination source 52 and/or detector 54.

Besides functioning as an optical filter, the support 21 may also posses other desirable optical qualities. As mentioned above, the support 21 may contain a mask, light guiding element, lens, diffuser, etc. For example, the support 21 may be a light diffuser formed from a polymeric film containing “white” titanium dioxide particles. In some cases, when employed in the support 21, it is desired that “micro-optic” elements are utilized. Micro-optic elements generally have a size less than about 2 millimeters and are arranged in one or two dimensions. Due to their small size, micro-optic elements may be more readily utilized in the support 21.

When the support 21 is optimized for a particular optical property, the material(s) used for forming the support 21 may be selected to possess the desired optical property. Alternatively, the desired optically functional material may simply be applied to the support 21 before and/or after forming the assay device 20. Such an optically functional material may be applied to the support 21 in a variety of ways. For example, the optically functional material may simply be dyed or coated onto one or more surfaces of the support 21. When applied in this manner, the optically functional material may cover only a portion or an entire surface of the support 21. In one embodiment, for example, the optically functional material is applied to a portion of the support 21 that corresponds to the detection zone 31 and/or calibration zone 32. In this manner, the optically functional material may enhance the detection or calibration signals emitted from the assay device 20 during use. Alternatively, the optically functional material may also be incorporated into the structure of the support 21. For example, internal optics may be formed using known techniques, such as embossing, stamping, molding, etc.

One embodiment of a method for detecting the presence or absence of an analyte using the optical detection system of the present invention will now be described in more detail. It should understood, however, that the description set forth below is for exemplary purposes only, and that other embodiments are contemplated by the present invention. Although other types of assays may be utilized, a sandwich-type immunoassay is referenced in this embodiment for purposes of illustration.

For example, referring again to FIG. 1, a test sample may initially be applied to sample pad (not shown) where it may travel to the conjugate pad 22. At the conjugate pad 22, the analyte (e.g., antigen) within the test sample forms complexes with fluorescent detection probes conjugated with a specific binding member (e.g., antibody) for the analyte. Thereafter, the complexed fluorescent probes travel to a detection zone 31 where they are captured by a receptive material contained therein. If desired, fluorescent calibration probes (may or may not be conjugated) may also be utilized that bind to a receptive material contained within a calibration zone 32. Once captured, the signal of the probes at the detection zone 31 and calibration zone 32 are measured using the fluorescence reader 50. In this particular embodiment, the illumination source 52 emits pulsed light so that time-resolved detection techniques may be employed. Likewise, the detector 54 is time-gated, meaning that it only detects the signal emitted by the excited fluorescent probes after a certain response time, which is typically from about 1 to about 200 microseconds.

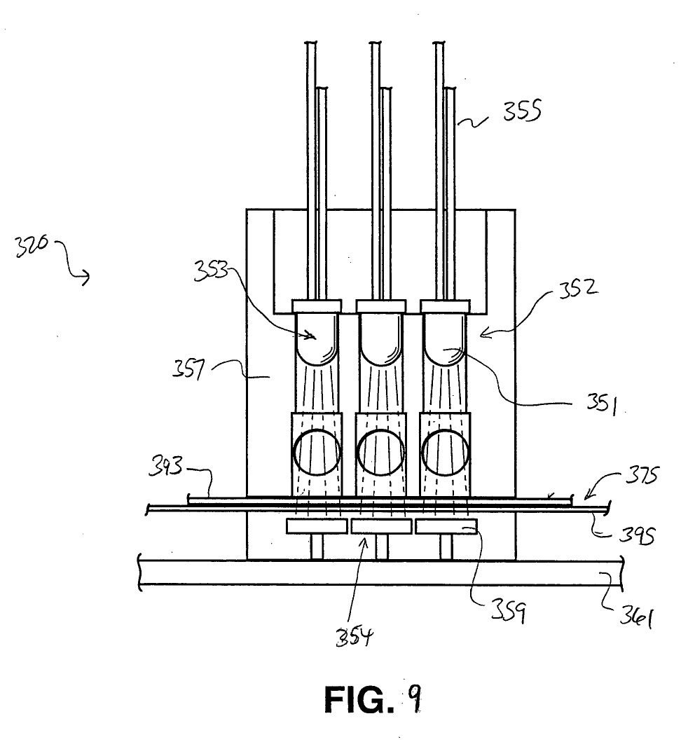

Various timing circuitry is used to control the pulsed excitation of the illumination source 52 and the time-gated measurement of the detector 54. For instance, referring to FIG. 4 exemplary timing circuitry that may be utilized in the present invention is shown. In this particular embodiment, a clock source 56 (e.g., a crystal oscillator) is employed to provide a controlled frequency source to other electronic components in the fluorescence reader 50. For instance, the oscillator 56 may generate a 20 MHz signal, which is provided to an LED driver/pulse generator 55 and to an A/D converter 64. The clock signal from oscillator 56 to A/D converter 64 controls the operating speed of A/D converter 64. It should be appreciated that a frequency divider may be utilized in such respective signal paths if the operating frequency of A/D converter 64 or if the desired frequency of the clock input to LED driver/pulse generator 55 is different than 20 MHz. Thus, the signal from oscillator 56 may be modified appropriately to provide signals of a desired frequency. In some embodiments, a signal from oscillator 56 may also be provided to microprocessor 60 to control its operating speed. Additional frequency dividers may be utilized in other signal paths in accordance with the present invention.

Microprocessor 60 provides control input to pulse generator 55 such that the 20 MHz signal from oscillator 56 is programmably adjusted to provide a desired pulse duration and repetition rate (for example, a 1 kHz source with a 50% duty cycle). The signal from pulse generator 55 may then be provided to the illumination source 52, controlling its pulse repetition rate and duty cycle of illumination. In some embodiments, a transistor may be provided in the signal path to the illumination source 52, thus providing a switching means for effecting a pulsed light signal.

As described above, the pulsed light excites the fluorescent probes located at the detection zones 31 and/or 32. After a desired response time, the detector 54 detects the signal emitted by the excited fluorescent probes and generates an electric current representative thereof. This electric current may then be converted to a voltage level by a high-speed transimpedance preamplifier 78, which may be characterized by a relatively low settling time and fast recovery from saturation. The output of the preamplifier 78 may then be provided to the data input of A/D converter 64. Additional amplifier elements (such as a programmable gain amplifier) may be employed in the signal path after preamplifier 278 and before A/D converter 64 to yield a signal within an appropriate voltage range at the trailing edge of the excitation pulse for provision to the A/D converter 64. A/D converter 64 may be a high-speed converter that has a sample rate sufficient to acquire many points within the fluorescence lifetime of the subject fluorescence labels. The gain of the preamplifier 78 may be set such that data values drop below the maximum A/D count (e.g., 2047 for a 12-bit converter) on the trailing edge of the excitation pulse. Data within the dynamic range of A/D converter 64 would then be primarily representative of the desired fluorescence signal. If the sample interval is short compared with the rise-time and fall-time of the excitation pulse, then the gain of preamplifier 78 may be set to ensure that signal values within the upper Y2 or ¾ of the dynamic range of A/D converter 78 correspond to the trailing edge of the emission pulse.

A/D converter 64 samples the signal from preamplifier 78 and provides it to the microprocessor 60 where software instruction is configured for various processing of the digital signal. An output from the microprocessor 60 is provided to the A/D converter 64 to further control when the detected fluorescence signal is sampled. Control signals to preamplifier 78 (not shown) and to A/D converter 64 may be continuously modified to achieve the most appropriate gain, sampling interval, and trigger offset. It should be appreciated that although the AID converter 64 and the microprocessor 60 are depicted as distinct components, commercially available chips that include both such components in a single module may also be utilized in the present invention. After processing, the microprocessor 60 may provide at least one output indicative of the fluorescence levels detected by the detector 54. One such exemplary output is provided to a display 86, thus providing a user with a visual indication of the fluorescence signal generated by the probes. Display 86 may provide additional interactive features, such as a control interface to which a user may provide programmable input to microprocessor 60.

Yet another embodiment of representative specific electronic components for use in the fluorescence reader 50 is illustrated in FIG. 5. Many of the components in FIG. 5 are analogous to those of FIG. 4 so the same reference characters are used in such instances. One difference in the reader 50 of FIG. 5 as compared to that of FIG. 4 is that the generation of a gate signal at phase delay module 57. A control signal from microprocessor 60 is provided to phase delay module 57 to program the effective phase shift of a clock signal provided thereto. This shifted clock signal (also referred to as a gate signal) is then provided to a mixer 58 where such signal is multiplied by the periodic detector signal received by the detector 54 and passed through preamplifier 78. The resulting output of mixer 58 is then sent through a low-pass filter 62 before being provided to A/D converter 64. A/D converter 64 may then measure the output of low-pass filter 62 to obtain a measurement of the fluorescence during intervals defined by the gate signal.

Still further alternative features for an exemplary fluorescent reader embodiment 50 are illustrated in FIG. 6. For instance, a sample/hold amplifier 88 (also sometimes referred to as a track-and-hold amplifier) is shown that captures and holds a voltage input signal at specific points in time under control of an external signal. A specific example of a sample/hold amplifier for use with the present technology is a SHC5320 chip, such as those sold by Burr-Brown Corporation. The sample/hold amplifier external control signal in the embodiment of FIG. 6 is received from a delay circuit 92, which may, for instance, be digital delay circuit that derives a predetermined delay from the clock using counters, basic logic gates, and a flip-flop circuit. Delay circuit 92 receives a clock signal from oscillator 56 and an enable signal from frequency divider 90, which simply provides a periodic signal at a reduced frequency level than that generated at oscillator 56. Delay circuit 92 may also receive a control input from microprocessor 60 to enable programmable aspects of a delay to ensure proper sampling at sample/hold amplifier 88. The delayed pulse control signal from delay circuit 92 to sample/hold amplifier 88 thus triggers acquisition of the fluorescence signal from the detector 54 at preset time intervals after the illumination source 52 has turned off.

Generally speaking, qualitative, quantitative, or semi-quantitative determination of the presence or concentration of an analyte may be achieved in accordance with the present invention. For example, in one embodiment, the amount of the analyte may be quantitatively or semi-quantitatively determined by correlating the intensity of the emitted signal, Iₛ, of the probes captured at the detection zone 31 with a predetermined analyte concentration. In some embodiments, the intensity of the signal, Iₛ, may also be compared with the intensity of the emitted signal, I_{c}, of the probes captured at the calibration zone 32. The intensity of the signal, Iₛ, may be compared to the intensity of the signal, I_{c}. In this embodiment, the total amount of the probes at the calibration zone 32 is predetermined and known and thus may be used for calibration purposes. For example, in some embodiments (e.g., sandwich assays), the amount of analyte is directly proportional to the ratio of Iₛ to I_{c}. In other embodiments (e.g., competitive assays), the amount of analyte is inversely proportional to the ratio of Iₛ to I_{c}. Based upon the intensity range in which the detection zone 31 falls, the general concentration range for the analyte may be determined. As a result, calibration and sample testing may be conducted under approximately the same conditions at the same time, thus providing reliable quantitative or semi-quantitative results, with increased sensitivity.

If desired, the ratio of Iₛ to I_{c} may be plotted versus the analyte concentration for a range of known analyte concentrations to generate a calibration curve. To determine the quantity of analyte in an unknown test sample, the signal ratio may then be converted to analyte concentration according to the calibration curve. It should be noted that alternative mathematical relationships between Iₛ and I_{c} may be plotted versus the analyte concentration to generate the calibration curve. For example, in one embodiment, the value of Iₛ/(Iₛ+I_{c}) may be plotted versus analyte concentration to generate the calibration curve.

The present invention may be better understood with reference to the following examples.

### EXAMPLE 1

The ability to form a luminescent detection system in accordance with the present invention was demonstrated. Luminescent detection probes were initially formed for use in the optical detection system. Specifically, 2.4 milligrams of an epoxy-functional terpolymer resin (Dow Chemical Co. of Midland, Mich. under the name UCAR™ VERR-40); 0.6 milligrams of a vinyl resin (available from Dow Chemical under the name UCAR™ VMCA); and 30 micrograms of platinum (II) tetra-meso-fluorophenylphorphine (Pt-TMFPP) (Frontier Scientific Inc. of Logan, Utah) were dissolved into 0.6 milliliters of tetrahydrofuran. 3 milliliters of water was then added to the mixture under vigorous stirring through a syringe pump with a delivery rate of 7 milliliters per minute. The particles were dialyzed three times in water to remove the tetrahydrofuran. Next, the particles were suspended in water to form a suspension (2 milligrams per milliliter) and heated at 80° C. for 3 hours to crosslink the particles. The particle size was determined to be 120 nanometers with a polydispersity of 0.05 ZetaPals (ZetaPotential analyzer from Brookhaven Instruments Co. of Holtsville, N.Y.). The resulting encapsulated particles exhibited very strong phosphorescence at an emission wavelength of 650 nanometers when excited at 390 nanometers under ambient conditions measured by Fluorolog III fluorimeter (Jobin Yvon, Inc. of Edison, N.J.).

A lateral flow strip was also formed for use in the detection system. Specifically, a nitrocellulose porous membrane (available from Millipore, Inc. of Bedford, Mass. under the name HF 1202) having a length of approximately 30 centimeters was laminated onto transparent polyester support cards made by GML Inc. of St. Paul, Minn. A cationic polyelectrolyte available from National Starch & Chemical, Inc. under the name CELQUAT® SC-230M (0.05 wt. %) was striped onto the membrane to form a detection zone. The membrane samples were then dried for 1 hour at a temperature of 37° C. A cellulosic fiber wicking pad (Millipore, Inc. Co.) was attached to one end of the membrane and cut into 4-millimeter half strips. Thereafter, nine wells were provided on a microtiter plate. Each well contained 40 microliters of hepes buffer (20 millimolar, pH 7.4) and Tween 20 (0.5%, Aldrich). In addition, different amounts of phosphorescent Pt-TMFPP particles were provided in each well, namely, 0, 8, 16, 32, 80,160, 320, 800 and 3200 nanograms. Lateral flow strips, such as described above, were then inserted into each well to capture the luminescent detection probes on the detection zone.

Thereafter, a luminescent reader such as shown in FIGS. 8 and 9 was utilized to detect the presence of the luminescent probes at the detection zone of each strip. Specifically, each of the lateral flow strips was laid down on a sample holder made of machined aluminum. The sample holder had a small window to allow light to pass through. The size of this window was chosen to restrict measurement to a small area surrounding the detection zone (to minimize the contribution of background scattering and fluorescence from the lateral flow device. The detection zone of the device was aligned with the window and the position of the device was secured using tape. The strips and sample holder were then inserted into a cartridge holder of a luminescent reader.

The reader was formed from a UV LED (Kingbright, 390 nanometers in size within a T-1 resin package) and a silicon photodiode (BWP-34) mounted on opposite sides of the sample carrier. The photodiode was placed approximately 1 millimeter from the support card lateral flow strip to maximize the phosphorescent flux collection solid angle. Between the photodiode and the sample carrier was placed a piece of Roscolux red filter (deep amber-E022 from Rosco Laboratories, Inc. of Stamford, Conn.) to minimize the detection of flux from sources other than phosphorescence. The LED was mounted on the opposite side of the carrier for the lateral flow strip to face the membrane. Specifically, the LED was mounted at the end of an aluminum tube approximately 1.5 centimeters in length. The tube was used to restrict illumination to only the area of interest. The electronic circuitry shown in FIG. 4 was used to flash the LED at a frequency of approximately 1 kHz, with a duty cycle of approximately 20%, at a drive current of approximately 30 mA, and with a fall-time to quench emission of less than 1 microsecond. A two stage amplifier consisting of a transimpedance amplifier (approximately 10,000 V/A) and a voltage amplifier (gain of approximately 100) was used to convert small detector photocurrents into easily measured voltages (approximately 1 volt) with sufficient bandwidth to measure phosphorescent decay with a sampling rate on the order of 250 kHz.

To perform the data analysis, a digitizing oscilloscope (A/D converter) was used to acquire 4096 point records with a sampling frequency of approximately 200 kHz. The N value of these records (typically 128) was averaged to obtain a result with a signal to noise ratio ˜\sqrt{N} times larger than that of a single record. The m^{th} value may be represented as the following sequence of samples:
x₀^{m}, x₁^{m}, . . . , xₙ^{m}, . . . x₄₀₉₄^{m}, x₄₀₉₅^{m}
The sequence is measure at times:
tₘ,tₘ+Δt, . . . , tₘ+nΔt, . . . , tₘ+4094Δt,tₘ+4095Δt
wherein, Δt is the sampling period (approximately 5 microseconds). Each of the starting times tₘ for the measurement of each record was positioned so that the measured waveforms had the same phase with respect to the LED drive signal for all x₀^{m}. The average of “N” records was computed as follows:

$${x}_{n} = \frac{1}{N} {\sum }_{m = 1}^{N} {x}_{n}^{m} .$$

Averaging of multiple periods contained within the average was possible in cases where the period was a large integer multiple of the sampling interval. For data analysis, discrete points xₙ for times tₙ of interest (e.g., tₙ=approximately half of the phosphorescence lifetime) were compared for specimens with different numbers of dyed particles. For less noise sensitive comparisons, an integral over a time window (for example from t₁=0.1 lifetime to t₂=0.4 lifetime) was constructed by summing data points falling within the window:

$${\int }_{{t}_{1}}^{{t}_{2}} w ( t ) ⅆ t \approx Δ t {\sum }_{n = {n}_{1}}^{{n}_{2}} {x}_{n}$$

wherein, t₁˜n₁Δt, t₂≈n₂Δt, and w(t) represents the ideal (non-discretized) averaged waveform.

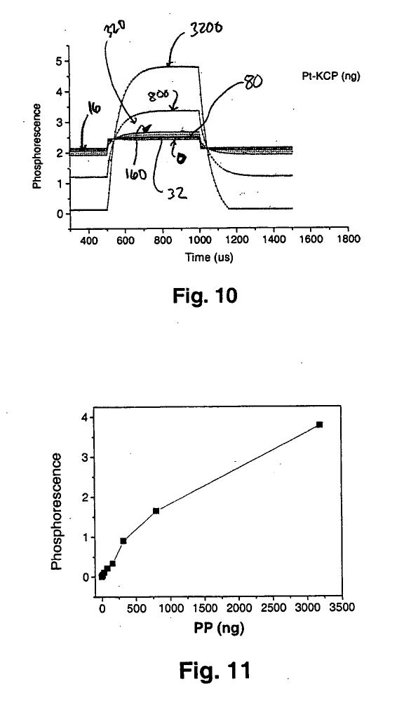

Using the technique described above, the phosphorescence response curve over time was then detected. FIG. 10 shows the time-resolved phosphorescence of the devices and FIG. 11 shows the dose response curve at a 40-microsecond time delay, corrected by the background phosphorescence at a 200-microsecond delay time. The data for FIGS. 10 and 11 were based on the averaging of 40 pulses.

### EXAMPLE 2

The ability to form a luminescent detection system in accordance with the present invention was demonstrated. Luminescent detection probes were initially formed for use in the optical detection system. Specifically, 2.4 milligrams of an epoxy-functional terpolymer resin (Dow Chemical Co. of Midland, Mich. under the name UCAR™ VERR-40); 0.6 milligrams of a vinyl resin (available from Dow Chemical under the name UCAR™ VMCA); and 30 micrograms of palladium (II) tetra-meso-fluorophenylporphine (Pd-TMFPP) (Frontier Scientific Inc. of Logan, Utah) were dissolved into 0.6 milliliters of tetrahydrofuran. 3 milliliters of water was then added to the mixture under vigorous stirring through a syringe pump with a delivery rate of 7 milliliters per minute. The particles were dialyzed three times in water to remove the tetrahydrofuran. Next, the particles were suspended in water to form a suspension (2 milligrams per milliliter) and heated at 80° C. for 3 hours to crosslink the particles. The particle size was determined to be 160 nanometers with a polydispersity of 0.09 ZetaPals (ZetaPotential analyzer from Brookhaven Instruments Co. of Holtsville, N.Y.). The resulting encapsulated particles exhibited very strong phosphorescence at an emission wavelength of 670 nanometers when excited at 390 nanometers under ambient conditions measured by Fluorolog III fluorimeter (Jobin Yvon, Inc. of Edison, N.J.).

A lateral flow strip was also formed for use in the detection system. Specifically, a nitrocellulose porous membrane (available from Millipore, Inc. of Bedford, Mass. under the name HF 1202) having a length of approximately 30 centimeters was laminated onto transparent polyester support cards made by GML Inc. of St. Paul, Minn.). A cationic polyelectrolyte available from National Starch & Chemical, Inc. under the name CELQUAT® SC-230M (0.05 wt. %) was striped onto the membrane to form a detection zone. The membrane samples were then dried for 1 hour at a temperature of 37° C. A cellulosic fiber wicking pad (Millipore, Inc. Co.) was attached to one end of the membrane and cut into 4-millimeter half strips. Thereafter, nine wells were provided on a microtiter plate. Each well contained 40 microliters of hepes buffer (20 millimolar, pH 7.4) and Tween 20 (0.5%, Aldrich). In addition, different amounts of phosphorescent Pd-TMFPP particles were provided in each well, namely, 0, 8,16, 32, 80, 160, 320, 800 and 3200 nanograms. Lateral flow strips, such as described above, were then inserted into each well to capture the luminescent detection probes on the detection zone.

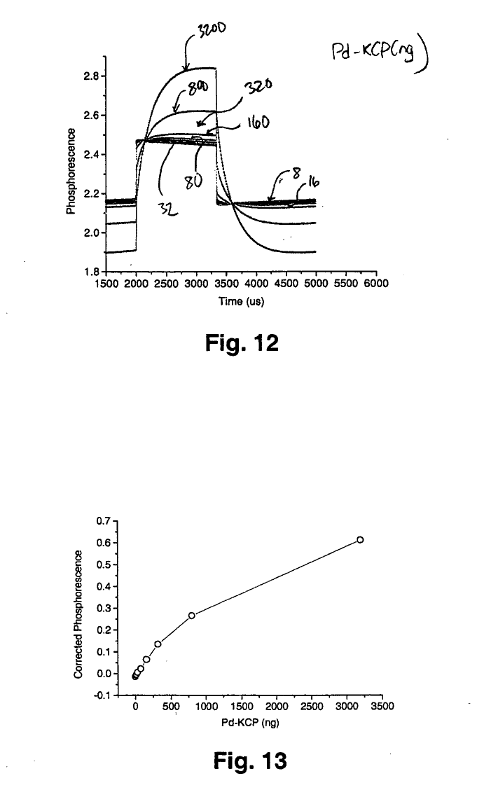

A luminescent detection system was then formed as described in Example 1, except that the LED was flashed at a frequency of approximately 0.3 kHz. FIG. 12 shows the time-resolved phosphorescence of the resulting devices and FIG. 13 shows the dose response curve at a 40-microsecond time delay, corrected by the background phosphorescence at a 200-microsecond delay time. The data for FIGS. 12 and 13 were based on the averaging of 40 pulses.

### EXAMPLE 3

A luminescent detection system was formed as described in Example 1, except that seven sets of sample strips were formed. Each strip was dipped into a well containing 40 microliters of hepes buffer (20 millimolar, pH 7.4) and Tween 20 (0.5%, Aldrich). In addition, different amounts of phosphorescent Pt-TMFPP particles were provided in each well, namely, 0, 0.62,1.3, 2.5, 5.0,10.0 and 20.0 nanograms. 10 duplicates were performed for each set of strips. Table 1 shows the average phosphorescence signal at a 40-microsecond delayed time for each series and its standard deviation.

TABLE 1
Phosphorescence Results

| Sample Set | 1 | 2 | 3 | 4 | 5 | 6 | 7 |
|---|---|---|---|---|---|---|---|
| Amount (ng) | 0.0 | 0.612 | 1.25 | 2.50 | 5.00 | 10.0 | 20.0 |
| I₄₀-I₂₀₀ | −9.1E⁻⁴ | −4.6E⁻⁴ | 6.9E⁻⁴ | 0.0024 | 0.0050 | 0.013 | 0.028 |
| Standard Deviation | 8.2E⁻⁴ | 7.4E⁻⁴ | 0.0011 | 0.0011 | 0.0011 | 0.0010 | 0.0032 |

Note:
I₄₀ and I₂₀₀ are the phosphorescence intensities at a 40- and 200-microsecond delayed time, respectively.

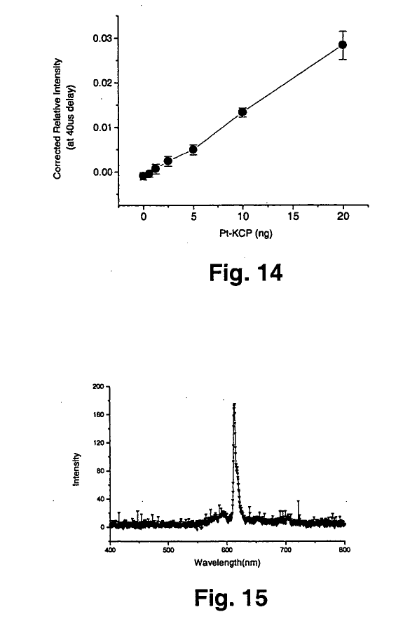

Likewise, FIG. 14 shows the dose response curve at a 40-microsecond time delay corrected by the background phosphorescence at a 200-microsecond delay time. Based on the criteria of signal of 2× standard deviation, the detection sensitivity was estimated to be 2.5 nanograms per device.

### EXAMPLE 4

The ability to form a luminescent detection system in accordance with the present invention was demonstrated. Specifically, a nitrocellulose porous membrane (available from Millipore, Inc. of Bedford, Mass. under the name HF 1202) having a length of approximately 30 centimeters was laminated onto translucent support cards made by Millipore, Inc. A cationic polyelectrolyte available from National Starch & Chemical, Inc. under the name CELQUAT® SC-230M (0.05 wt. %) was striped onto the membrane to form a detection zone. The membrane samples were then dried for 1 hour at a temperature of 37° C. A cellulosic fiber wicking pad (Millipore, Inc. Co.) was attached to one end of the membrane and cut into 4-millimeter half strips. Thereafter, a microtiter well was provided that contained 0.5 micrograms of carboxylated europium chelate-encapsulated particles available from Molecular Probes, Inc. (0.2-micrometer size, 0.5% solids content) in 40 microliters of 1% Tween 20 (Aldrich). The europium-based particles were captured on the detection zone. The developed device was then air-dried at room temperature for one hour.

The strip was then mounted on a sample holder for a Fluorolog III fluorimeter obtained from Jobin Yvon, Inc. of Edison, N.J. The membrane side of the strip faced the excitation lamp (selected at 380 nanometers by a monochromator) inside the fluorimeter, which was focused on the detection zone. The head of an optic fiber connected with an ocean optic detector (Ocean Optics, Inc. of Dunedin, Fla.) was positioned close to the support card side of the strip, near the detection zone. Between the optic fiber head and the detection zone was laid a cut-off 550-nanometer optical filter (Andover Co. of Salem, N.H.). FIG. 15 shows the resulting fluorescence spectrum.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A luminescent detection system for detecting the presence or quantity of an analyte residing in a test sample, said system comprising:
an assay device that includes a chromatographic medium, said chromatographic medium being in communication with luminescent detection probes, said luminescent detection probes being capable of emitting a detection signal;
an illumination source capable of providing electromagnetic radiation that excites said luminescent detection probes to emit said detection signal; and
a detector capable of registering said detection signal emitted by said luminescent detection probes, wherein said illumination source and said detector are positioned on opposing sides of said assay device so that said chromatographic medium is positioned in the electromagnetic radiation path defined between said illumination source and said detector, said chromatographic medium being transmissive to said electromagnetic radiation and said detection signal.

2. The luminescent detection system of claim 1, wherein said chromatographic medium is a porous membrane.

3. The luminescent detection system of claim 1, wherein a receptive material is immobilized within a detection zone defined by said chromatographic medium, said receptive material being configured to bind to at least a portion of said detection probes or complexes thereof.

4. The luminescent detection system of claim 1, wherein said detection probes comprise a particle labeled with a luminescent compound.

5. The luminescent detection system of claim 1, wherein said chromatographic medium is carried by a support.

6. The luminescent detection system of claim 5, wherein said illumination source is positioned adjacent to said chromatographic medium, and said detector is positioned adjacent to support.

7. The luminescent detection system of claim 5, wherein said illumination source is positioned adjacent to said support and said detector is positioned adjacent to said chromatographic medium.

8. The luminescent detection system of claim 1, further comprising an optical filter having a high transmissibility at one or more wavelengths specific to the electromagnetic radiation provided by said illumination source.

9. The luminescent detection system of claim 1, further comprising an optical filter having a high transmissibility at one or more wavelengths specific to said detection signal.

10. The luminescent detection system of claim 1, wherein said illumination source contains a light-emitting diode.

11. The luminescent detection system of claim 1, wherein said illumination source contains an electroluminescent device.

12. The luminescent detection system of claim 1, wherein said detector contains an electronic imaging sensor capable of spatial discrimination.

13. The luminescent detection system of claim 1, wherein said detector contains a photodiode.

14. The luminescent detection system of claim 1, further comprising timing circuitry in communication with said illumination source and said detector, said timing circuitry facilitating the provision of pulsed electromagnetic radiation by said illumination source and time-gated detection by said detector.

15. The luminescent detection system of claim 1, wherein said illumination source, said detector, or both, are positioned less than about 5 millimeters from said assay device.

16. The luminescent detection system of claim 1, wherein said illumination source, said detector, or both, are positioned less than about 2 millimeters from said assay device.

17. A luminescent detection system for detecting the presence or quantity of an analyte residing in a test sample, said system comprising:
an assay device that includes a porous membrane carried by a support, said porous membrane being in communication with luminescent detection probes, said luminescent detection probes being capable of emitting a detection signal;
an illumination source capable of providing pulsed electromagnetic radiation that excites said luminescent detection probes to emit said detection signal; and
a time-gated detector capable of registering said detection signal emitted by said luminescent detection probes, wherein said illumination source and said detector are positioned on opposing sides of said assay device so that said porous membrane is positioned in the electromagnetic radiation path defined between said illumination source and said detector, said porous membrane and said support being transmissive to said electromagnetic radiation and said detection signal.

18. The luminescent detection system of claim 17, wherein said illumination source is positioned adjacent to said porous membrane and said detector is positioned adjacent to said support.

19. The luminescent detection system of claim 17, wherein said detector is positioned adjacent to said porous membrane and said illumination source is positioned adjacent to said support.

20. The luminescent detection system of claim 17, wherein said light source contains a light-emitting diode.

21. The luminescent detection system of claim 17, wherein said light source contains an electroluminescent device.

22. The luminescent detection system of claim 17, wherein said detector contains a photodiode.

23. The luminescent detection system of claim 17, wherein said illumination source, said detector, or both, are positioned less than about 5 millimeters from said assay device.

24. The luminescent detection system of claim 1, wherein said illumination source, said detector, or both, are positioned less than about 2 millimeters from said assay device.

25. A method for detecting the presence or quantity of an analyte residing in a test sample with an assay device, the assay device comprising a porous membrane that defines a detection zone, the porous membrane being in communication with luminescent detection probes, said method comprising:
i) contacting the assay device with the test sample, wherein at least a portion of the luminescent detection probes become immobilized within the detection zone;
ii) pulsing electromagnetic radiation onto the detection zone, thereby exciting the luminescent detection probes to emit a detection signal that is transmitted through the porous membrane; and
iii) measuring the intensity of said transmitted detection signal.

26. The method of claim 25, wherein the amount of the analyte within the test sample is proportional to the intensity of said transmitted detection signal.

27. The method of claim 25, wherein the porous membrane further comprises a calibration zone within which a calibration signal is capable of being generated, the amount of the analyte within the test sample being proportional to the intensity of said transmitted detection signal as calibrated by the intensity of said calibration signal.

28. The method of claim 25, wherein a period of time elapses between a pulse of said electromagnetic radiation and measurement of said detection signal.

29. The method of claim 28, wherein said period of time is from about 1 to about 200 microseconds.

30. The method of claim 28, wherein said period of time is from about 10 to about 50 microseconds.
